(19) United States
(12) Patent Application Publication
(10) Pub. No.: US 2022/0349881 A1
(43) Pub. Date: Nov. 3, 2022

(54) IN VIVO AND POINT-OF-CARE SAMPLE CAPTURE AND TESTING SYSTEMS

(71) Applicant: Duke University, Durham, NC (US)

(72) Inventors: Brad BARNETT, Durham, NC (US); Sharon FEKRAT, Durham, NC (US); Thomas DENNY, Durham, NC (US)

(21) Appl. No.: 17/763,845

(22) PCT Filed: Sep. 28, 2020
(86) PCT No.: PCT/US20/53103

§ 371 Date: Mar. 25, 2022

Related U.S. Application Data
(60) Provisional application No. 62/906,989, filed on Sep. 27, 2019.
(60) Provisional application No. 63/024,123, filed on May 13, 2020.

(51) Int. Cl. G01N 33/543; G01N 33/569; G01N 21/77

(52) U.S. Cl. CPC G01N 33/54373; G01N 33/56983; G01N 21/7703; A61B 5/153

(57) ABSTRACT

A sample capture and testing system can include a primary receptacle for receiving a fluid sample, where the primary receptacle has a sensor for label-free detection of an analyte disposed at one side. The primary receptacle can be incorporated in a needle for in vivo capture and testing of a sample. The primary receptacle can be incorporated into a specimen container and used as part of a point of care device.

### CROSS-REFERENCE TO RELATED APPLICATIONS

The present invention claims the benefit of U.S. Provisional Application Ser. No. 62/906,989, filed Sep. 27, 2019, and U.S. Provisional Application Ser. No. 63/024,123, filed May 13, 2020, which are hereby incorporated by reference in its entirety, including any figures, tables, and drawings.

### BACKGROUND

There are many methods of determining analytes that require post-processing of sample. For samples that pose a health risk to the end-user and in instances in which a sample needs to be processed rapidly by an end-provider in a point of care (POC) setting, automated systems that preclude post-processing are advantageous. These automated POC systems could be employed in various field medical and defense settings as well as broadly in medical and veterinary settings and even by the lay person for at home and in office testing. In order for a POC system that does not require post-processing, the necessary components of the assay must be included in the specimen collection reservoir or in a probe inserted into the patient. This in turn enables, sample testing that does not require for the end-user to expose themselves to the sample.

A potential means of closed-system analyte measurement would be to incorporate a biosensor directly into a specimen container or within a probe that may be inserted into a remote location. Examples of potential biosensors include plasmon resonance (SPR), localized surface plasmon resonance (LSPR), Mach-Zehnder interferometric (MZI), ring resonator and bio-layer interferometry (BLI). BLI detects biomolecular interactions between analyte and non-analyte pairs. A surface of a diagnostic lens is coated with one component of the pair and exposed to an environment containing the second component. Light is directed through the lens, and a reflection pattern is used to measure change in surface thickness over time, thus determining the presence and concentration of the second component.

However, a drawback of this approach is that the analyte to be measured must be analyzed ex vivo in a controlled laboratory setting. It is therefore difficult to measure some types of components, such as components that are difficult to access, components with low volume or low concentration, or components which may change in nature upon removal from the source. It is also difficult to use this equipment at a point of care.

A means of sensitive in vivo detection of analytes would therefore be advantageous from a diagnostic and therapeutic standpoint. In addition, it is desirable to conduct a point of care diagnostic that can be accomplished without the preprocessing and controls required for such a method of determining chemical analytes.

### BRIEF SUMMARY

In vivo and point-of-care sample capture and testing systems are described. In addition to rapid measurement of infectious agents, such systems could be used for the measurement of an endless number of analytes in a POC setting and even at home by the end-user. Similar to the digital home glucometer or the lateral flow assay (LFA) point of care pregnancy test, closed-system testing can have profound impact in the medical setting and beyond. Various embodiments of the described systems may bring the precision afforded by more advanced diagnostic techniques requiring lab-based manipulation by a skilled practitioner together with the simplicity of use characteristic of glucometer and LFA assays to enable these complex advanced diagnostics to be employed at a rate and scale previously unachievable. Such systems are particularly useful to address the current COVID-19 outbreak as well as for future pandemics.

A sample capture and testing system can include a primary receptacle for receiving a fluid sample, where the primary receptacle has a sensor for label-free detection of an analyte. Examples of analytes include protein, DNA, RNA, element, chemical, lipid, carbohydrate or any other analyte that will conjugate to a capture (“binding”) moiety. As clear to one skilled in the art, any of these analytes conjugated to another analyte will enable simultaneous detection of the conjugated analyte, for example antibody binding to a surface protein expressed on a virus will enable detection of the virus.

In one embodiment, the sensor for the sample capture and testing system is a biosensor that can detect one antigen or a plurality of antigens. For example, the biosensor can have a moiety that binds to a specific antigen on a virus.

In some cases, the system can further include a second sensor for label-free detection of a second analyte disposed at a side of the primary receptacle. For example, the analyte detected by the first sensor can be a target analyte of SARS-CoV-2 and the second analyte detected by the second sensor can be a target analyte of a flu virus. Each sensor can include associated corresponding analytes (or specific antibody) for detecting presence of a relevant virus.

A secondary reagent may be included in the primary receptacle or in a secondary receptacle (e.g., for cases where the second sensor is disposed at a side of a secondary receptacle instead of the primary receptacle) to improve signal capture. The secondary reagent may include a combination of materials, for example an antibody or other moiety that binds to the analyte of interest. To increase the signal, the antibody or other binding moiety can be conjugated to a nanoparticle. Examples of potential nanoparticles include, but are not limited to, carbon-based nanoparticles, ceramic nanoparticles, quantum dots, silica nanoparticles, micelles, dendrimers, liposomes, metal nanoparticles, semiconductor nanoparticles, polymeric nanoparticles including nanospheres and nanocapsules, lipid-based nanoparticles, carbon nanotubes (CNTs) and fullerenes.

As clear to one skilled in the art, the primary capture moiety at the sensor and the secondary capture moiety (e.g., as the secondary reagent) can be freely exchanged. If a plurality of the same antigen is present on the analyte of interest, for example in the case of virus, the capture antibody and the secondary antibody may be the same. In the event the analyte of interest only has a single moiety to which the capture moiety can bind, the primary capture moiety and the secondary capture moiety can be used to recognize non-competing regions on the analyte of interest.

In some cases, a sample capture and testing system can include a specimen container holder; a plurality of sensor tips; a coupler comprising a housing that receives a sensor tip of the plurality of sensor tips; and a bar configured to couple the sensor tip to a remaining component of a sensor and computing system, the bar further configured to apply pressure to the sensor tip to push the sensor tip into the specimen container holder. In some cases, the system can further include a sensor chamber configured to hold the plurality of sensor tips to be fed to the coupler; a sensor tip advancer that causes the sensor tip of the plurality of sensor tips to be advanced to the coupler; and, optionally, a disposal container configured to capture the sensor tip after use.

In some cases, the system can enable reuse of sensor tips by including at least one regeneration station. Regeneration of the probe may occur through the use of a dissociation buffer that causes the dissociation of the diagnostic binding pair. In one embodiment, the regeneration station of the system consists of a dissociation buffer station alone. In another embodiment, the regeneration station of the system includes a dissociation buffer followed by a rinse buffer station. In order to facilitate repetitive regeneration, a carousel is provided that is configured to hold the plurality of sensor tips and rotate the plurality of sensor tips from the specimen container holder sequentially to a buffer station, then to a rinse station (and to any other buffer and/or rinse stations) and return to the specimen container holder as the bar is applied in subsequent operations.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIGS. 1A-1C illustrate a sample and testing system in accordance with various embodiments of the invention.

FIGS. 2A-2E illustrate representations of label-free technologies that may be used in the described systems and devices.

FIG. 3A depicts an example embodiment of a BLI probe.

FIG. 3B depicts an embodiment of an in vivo BLI probe within a needle shaft.

FIG. 3C illustrates interaction between a sensor probe and SARs-CoV-2.

FIG. 3D illustrates a prototype configuration used for testing proof of principle for SARS-CoV-2 materials.

FIG. 4A and 4B show an example point of care sample capture and testing device.

FIG. 4C shows an implementation of a sample capture and testing device with reusable sensor tips.

FIG. 4D shows a plot of concentration over time.

FIGS. 5A and 5B illustrate various multiplex options.

FIG. 5C illustrates a multiplex option with secondary antibody/antibody nanoparticle in a capillary reservoir.

FIGS. 6A and 6B illustrate sensor tips integrated in a sample capture tube.

FIG. 6C illustrates a serum separator configuration.

FIG. 7 shows a plot of concentration to binding rate.

FIG. 8 shows a representation of biosensor capture for sensor testing of AHC (anti-human Fc), ProA (Protein A), and AR2G (amine coupling) with respect to capture of Cov2 Abs.

FIGS. 9A-9E show testing with BLI-AHC sensors.

FIGS. 10A-10E show testing with BLI-ProA sensors.

FIGS. 11A-11D show testing with BLI-AR2G sensors.

FIGS. 12A-12C show titration data for BLI-AHC Ab026065 binding response to nCoV-2 HexaPro.

FIGS. 13A-13I show plots for a titration dataset for nCoV-1 nCoV-2P on NTD Ab026016 mAb and for nCoV-1 nCoV-2P sandwich on NTD Ab026016 and RBD Ab026124 mAb.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

In vivo and point-of-care sample capture and testing systems are described. In addition to rapid measurement of infectious agents, such systems could be used for the measurement of an endless number of analytes in a POC setting and even at home by the end-user. Similar to the digital home glucometer or the lateral flow assay (LFA) point of care pregnancy test, closed-system testing can have profound impact in the medical setting and beyond. Various embodiments of the described systems may bring the precision afforded by more advanced diagnostic techniques requiring lab-based manipulation by a skilled practitioner together with the simplicity of use characteristic of glucometer and LFA assays to enable these complex advanced diagnostics to be employed at a rate and scale previously unachievable. Such systems are particularly useful to address the current COVID-19 outbreak as well as for future pandemics.

The COVID-19 outbreak has brought an unprecedented attention to the role of diagnostics testing of the causative virus, SARS-CoV-2. With the exception of SARS-CoV-1 and Middle East respiratory syndrome (MERS) and now SARS-CoV-2, human coronaviruses (HCoV) typically causes mild upper respiratory tract infections in humans. Prior to the introduction of these potentially fatal HCoV infections, other HCoV strains have widely circulated in the human population, including HCoV-229E, HCoV-0C43, HCoVHKU1, and HCoVNL63. As a whole, HCoV strains are highly transmissible and constitute a significant portion of common colds in persons of all ages.

The described sample capture and testing systems can be used for rapid processing and diagnosing of viruses including SARS-CoV-2.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

Unless otherwise defined, all technical terms used herein have the same meaning as commonly understood by one of ordinary skill in the art to which this disclosure belongs. As defined herein, a “bodily system” is any cavity, space, region, organ, etc. of a subject in which it is desired to detect a biological substance, also known as an “analyte”. There can be many purposes for measuring an analyte. Some of these include, for example, detection of viruses (e.g., SARS-CoV-2, HSV, VZV, CME, Zika, Chikungunya, and West Nile, among others) or bacteria (e.g., staph, strep, TB, syphilis, Lyme, toxoplasmosis, Toxocara, and histoplasmosis, among others). Further analytes that are useful to detect and/or measure include vascular endothelial growth factor, angiopoietin 2, interleukins, oxygen levels, and topical, injectable, and other medications. It may also be possible to analyze white blood cells and red blood cells, as well as other substances that will be apparent to those of skill in the art.

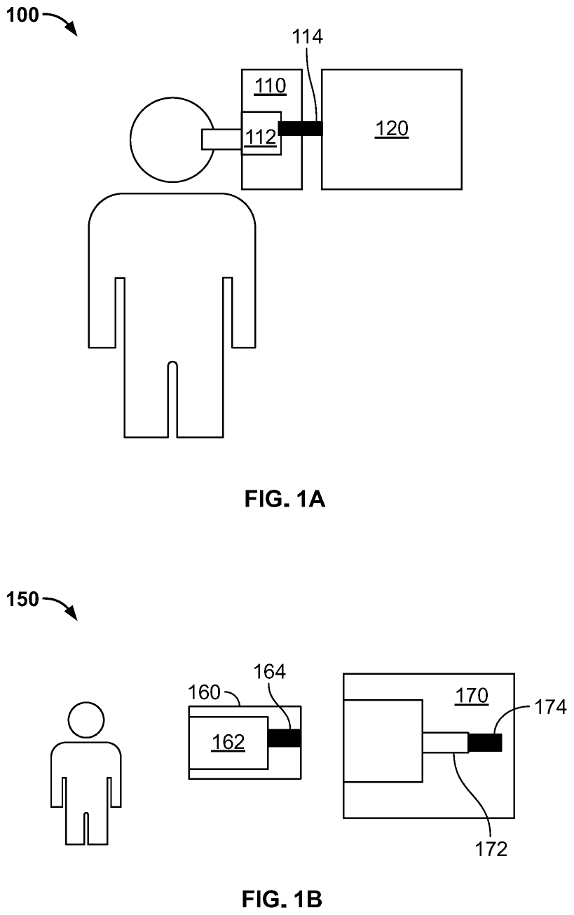

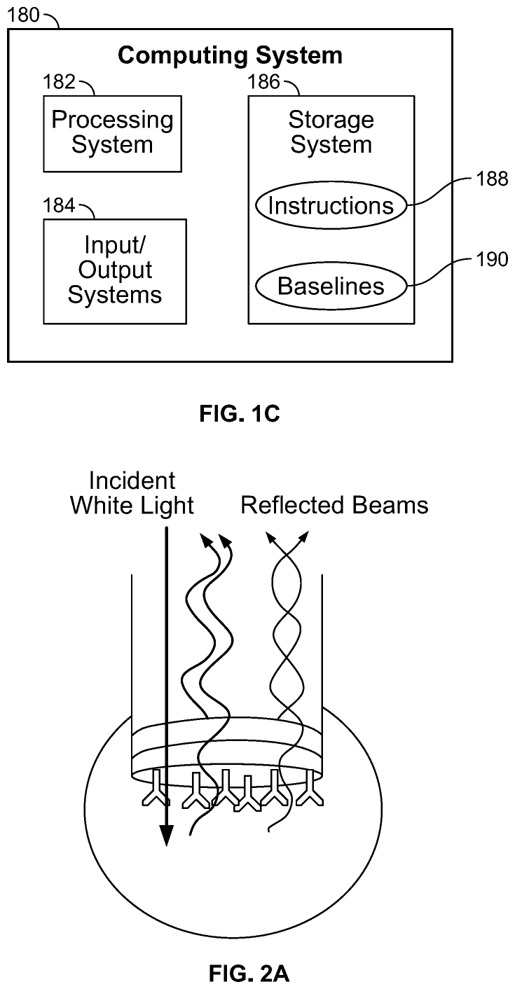

FIGS. 1A-1C illustrate a sample and testing system in accordance with various embodiments of the invention. Referring to FIG. 1A, a sample capture and testing system 100 can include a sample capture component 110 and a sample testing component 120. The sample capture component can include a primary receptacle 112 for receiving a fluid sample and a sensor 114 for label-free detection of a virus disposed at one side of the primary receptacle. The sample capture component 110 can be configured for in vivo application, where the sample capture component is implemented in the form of an oral probe, a nasal probe, an ocular surface probe, a respiratory bronchiole detection unit, or in a GI tract with fiber optic cable in endoscope working channel, as some examples. The sample testing component 120 can include components that are housed with the sample capture component or disposed separate but in communication with the sample capture component. The sample testing component 120 analyzes results of the sensor(s) at the sample capture component 110 and/or includes parts of the sensors (e.g., fiberoptic cables that couple to a probe tip of the sensor, spectrometer for measuring reflected light in a BLI-based implementation). In some cases, the sample testing component 120 can include or communicate with a computing system.

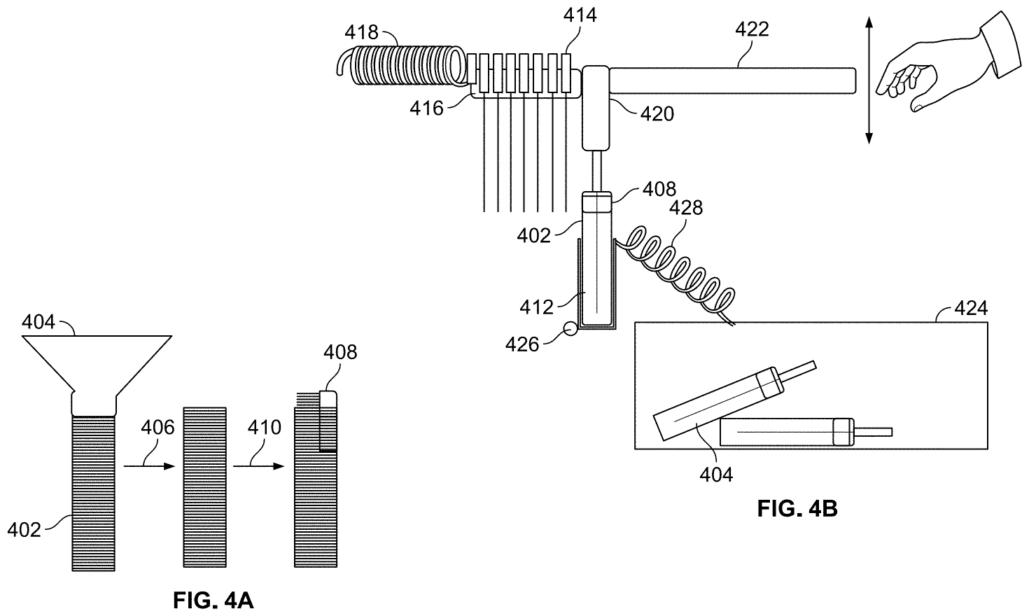

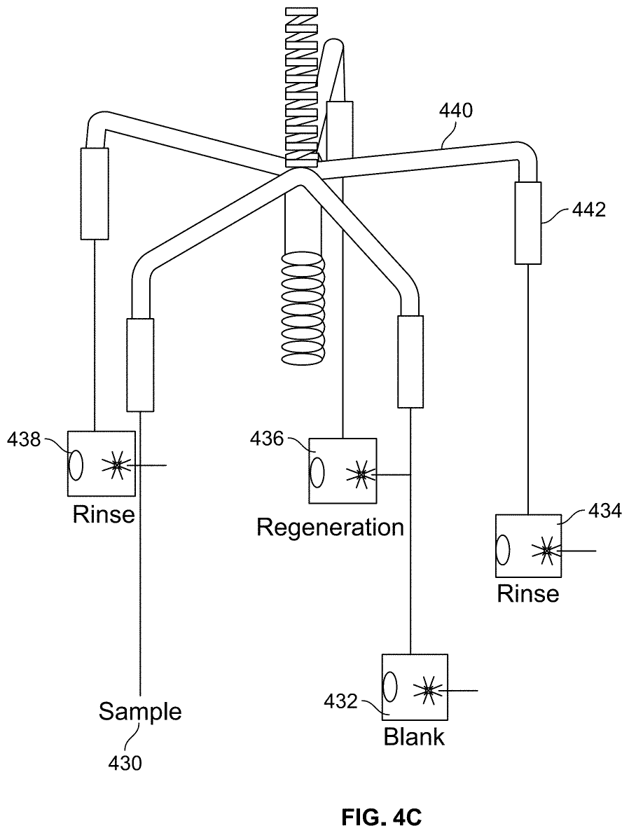

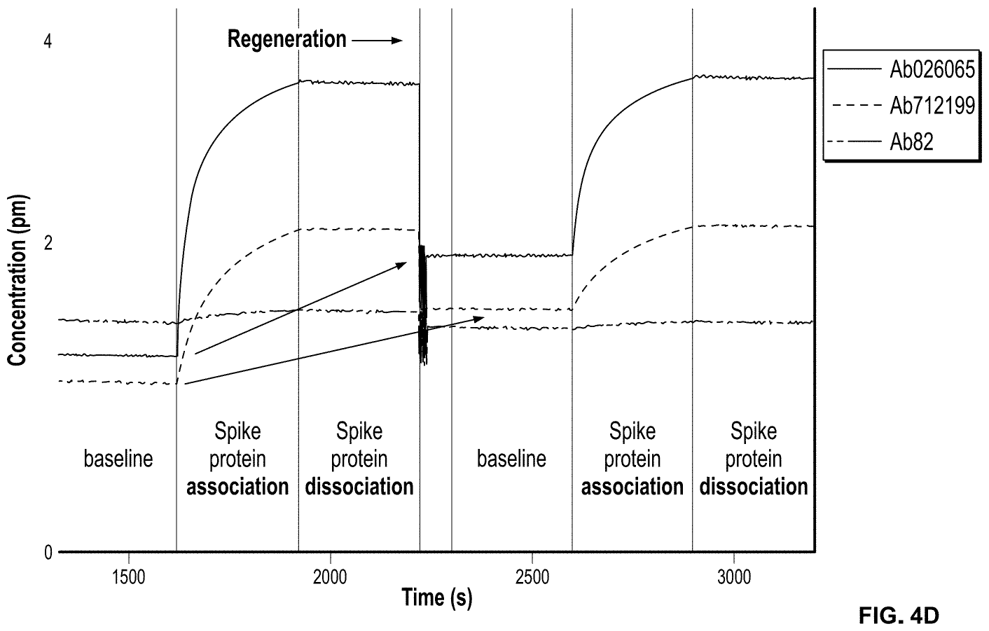

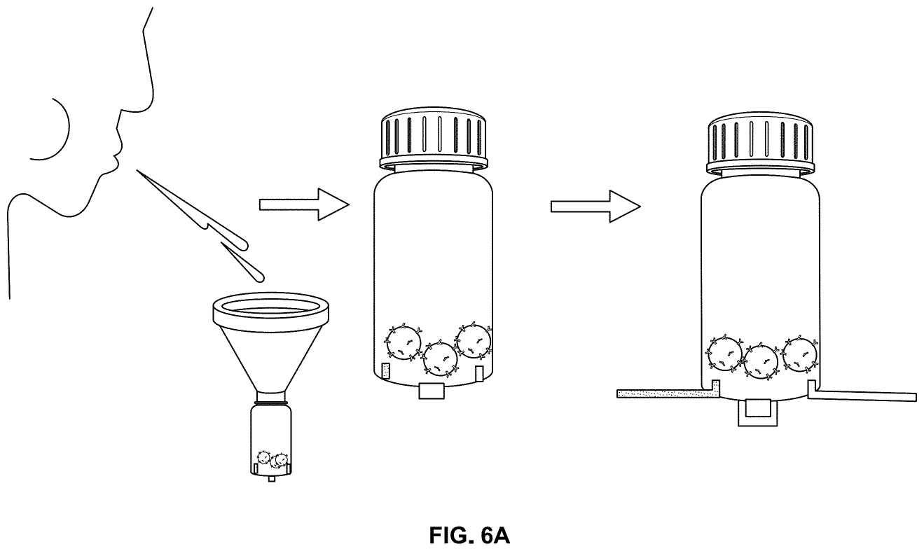

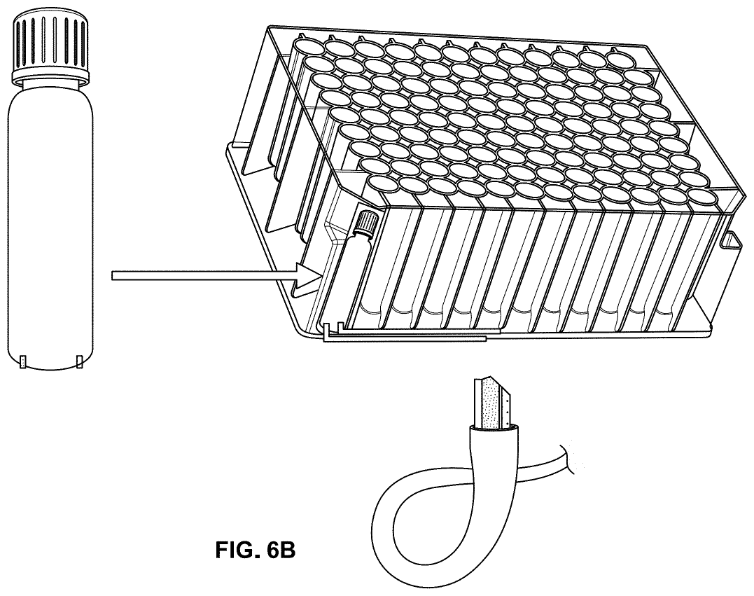

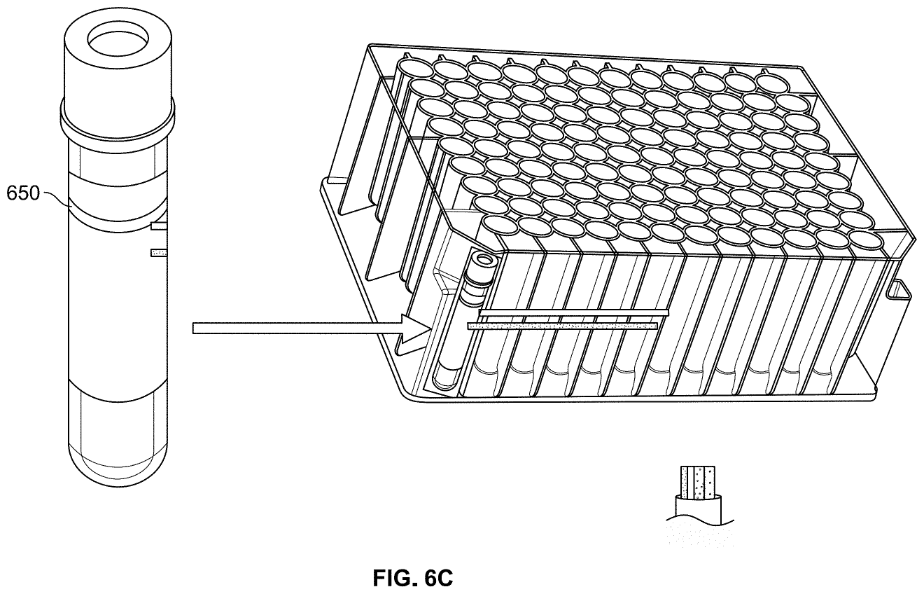

Referring to FIG. 1B, a sample capture and testing system 150 can include a sample capture component 160 and a sample testing component 170. The sample capture component can include a primary receptacle 162 for receiving a fluid sample. In some cases, the sample capture component includes a sensor 164 for label-free detection of a virus disposed at one side of the primary receptacle. In some cases, the sample testing component 170 includes a primary receptacle 172 for receiving a fluid sample from the sample capture component 160 and a sensor 174 for label-free detection of a virus disposed at one side of the primary receptacle of the sample testing component 170. The sample capture component 160 and sample testing component 170 can be configured for point of care testing (see e.g., examples of FIGS. 4A-4C and FIGS. 6A-6C).

Referring to FIG. 1C, a computing system 180 of a sample and testing system can include a processing system 182, input/output systems 184, and a storage system 186, which can store instructions 188 and baseline sensor data 190. The processing system 182 can include any suitable hardware processor such as a microprocessor, CPU, or GPU. The input/output systems 184 can include communication interfaces for network communication (wired or wireless), user interfaces (e.g., for input devices such as keyboard, touchscreen, and camera, and microphone, and output devices such as displays and speakers), and/or sensor interfaces (e.g., for receiving digital or analog signals from the appropriate sensor technologies). The storage system 186 can include any suitable storage devices, which may be in the form of volatile memory and/or nonvolatile memory. It should be understood that as used herein a “computer-readable storage medium” or “storage device” does not consist of transitory propagating signals.

The instructions 188 stored on a storage device of the storage system 186 can, when executed by the processing system 182, direct the computing system to obtain a baseline measurement for a sensor; store the baseline measurement in the storage device (e.g., as baseline sensor data 190); obtain a sample measurement from the sensor; compare the sample measurement with the baseline measurement; and output an indication of presence or absence of the virus to a display. In some cases, the baseline measurement is from historical captured data. In some cases, the baseline measurement is obtained from the sensor before obtaining the sample measurement from that sensor. Other instructions can be included, depending on the implementation.

Alternatively, or in addition, the functionality, methods, and processes described herein can be implemented, at least in part, by one or more hardware modules (or logic components) in place of or in addition to the processing system 182 and instructions 188. For example, the hardware modules can include, but are not limited to, application-specific integrated circuit (ASIC) chips, field programmable gate arrays (FPGAs), system-on-a-chip (SoC) systems, complex programmable logic devices (CPLDs) and other programmable logic devices now known or later developed. When the hardware modules are activated, the hardware modules perform the functionality, methods and processes included within the hardware modules.

Label-Free Technology Implementations

Label-free technologies involve real-time interaction between immobilized receptors and analytes without labelling (e.g., using radio or fluorescent labels) of the analyte.

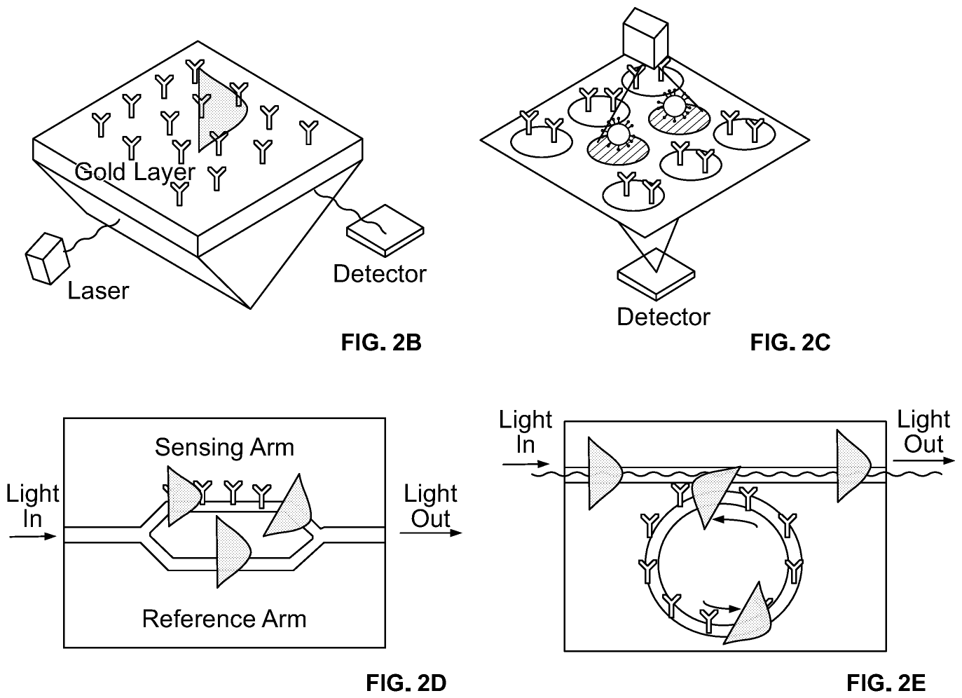

FIGS. 2A-2E illustrate representations of label-free technologies that may be used in the described systems and devices. FIG. 2A illustrates BLI. FIG. 2B illustrates surface plasmon resonance (SPR) biosensor in Kretschmann configuration. FIG. 2C illustrates a localized surface plasmon resonance (LSPR) biosensor based on gold nanodisks. FIG. 2D illustrates a Mach-Zehnder interferometric (MZI) biosensor. FIG. 2E illustrates a ring resonator biosensor.

Referring to FIG. 2A, a BLI probe has at least one pair of optic fibers or other similar light-transmitting medium, which form a light pathway with the optical element. One of the optic fibers transmits a light beam through the optical element, while the second fiber of the pair detects the reflected beam. The optic fibers extend from the distal end of the probe to a control portion of the device, where the wavelength changes are analyzed to determine the state of analyte pair binding.

One example of such a system includes fiber optic probes, in particular, employing a ligand on a fiberoptic tip for detection of an analyte. U.S. Pat. No. 8,305,585 describes Bio layer interferometry (BLI) for ex vivo analysis of samples. The sensing mechanism described therein can be used in certain embodiments of the systems described herein. For example, as explained in U.S. Pat. No. 8,305,585, one method involves reacting a sample solution with a first reflecting surface formed by a layer of analyte-binding molecules carried on the distal surface of a transparent optical element having a thickness of at least 50 nm, thereby to increase the thickness of the first reflecting layer by the binding of analyte to the analyte-binding molecules in the layer. The change in thickness of the first reflecting layer is measured by detecting a shift in a phase characteristic of the interference wave formed by the two light waves reflected from the first layer and from a second reflecting layer that is formed on the opposite, proximal surface of the optical element and which has an index of refraction greater than that of the optical element. The detecting step can include directing light from an optical fiber onto the two reflecting surfaces and directing reflected light from the two surfaces onto a detector through an optical coupling. The detector can be a spectrometer, where the detecting includes measuring a shift in the spectral position of one or more of the interference extrema produced by the two reflecting light waves. Where the method is used for measuring the rate of association of analyte to the second layer, the reacting step can be carried out until a near-maximum increase in thickness of the first reflecting layer is observed. Where the method is used for measuring the rate of dissociation of analyte to the second layer, the reacting steps can include immersing the second layer in a dissociation buffer for a period of time until a decrease in thickness of the first reflecting layer is observed. Where the method is used for measuring the amount of analyte present in the sample, the detecting is carried out over a period sufficient to measure the thickness of the first reflecting layer at a plurality of different time points. Where the method is used measuring one or more of a plurality of analytes in a sample, the first reflecting layer is composed of an array of discrete analyte-binding regions, the different regions being effective to bind different analytes, and the detecting is effective to detect a change in the thickness of each of the regions resulting from binding of analyte to the analyte-binding molecules.

BLI enables label-free detection of biomolecular interactions. BLI can detect the interference pattern of white light reflected from a layer of immobilized ligand on a biosensor tip, and an internal reference layer. The more analyte that binds to ligand on the biosensor tip causes the interference pattern to shift which enables real-time detection. As analyte from the solution in question binds to the ligand immobilized on the tip of the probe, the optical thickness adjusts thereby creating a wavelength shift, Δα. As Δλ directly correlates to the thickness of the biological layer, this enables precise measurement of analyte concentration. In addition to analyte concentration, as the interactions between ligand and analyte are measured in real time, rates of association as well as binding specificity can also be determined. Since unbound molecules do not impact the Δλ, a BLI probe can be used for in vivo detection of analytes.

Referring to FIG. 2B, a SPR biosensor generally employs a thin gold layer (e.g., 40-50 nm) as a transducer. An incident light beam excites coherent oscillations of the gold metal conduction band electrons (i.e., surface plasmon polariton) that propagates along the interface metal-dielectric, generating an evanescent field that can extend up to a few hundreds of nanometers (10-300 nm) into the surrounding medium. When specific biorecognition elements (e.g., antibodies or DNA strands) are immobilized onto the gold surface, the selective capture and binding of the target molecule induce a change of the refractive index and in the light properties, which can be monitored and is directly proportional to the concentration of the analyte in the sample. This propagating SPR uses a complex light coupling scheme (e.g., the prism-based Kretschmann configuration shown in the Figure).

Referring to FIG. 2C, an LSPR biosensor can be formed from a patterned surface with a nanodisk geometry. Other geometries include arrays of nanoholes, nanodisks, or nanorods, nanostars, nanodimers, and oligomer assemblies. Instead of propagating SPR, the patterned nanostructures exhibit a localized resonance (i.e., localized surface plasmon resonance, LSPR) that is characterized by the higher confinement of the evanescent field, with penetration depths around 10-50 nm, and by the spectral tunability of the resonance.

Referring to FIG. 2D, for a Mach-Zehnder (MZ), Young, or bimodal waveguide (BiMW) interferometer, an input waveguide is split into two arms (sensing and reference arms) that, after certain distance, are recombined into a single output. Biomolecular interactions occurring at the sensing arm within the evanescent field induce a phase difference with the light traveling along the reference arm. When recombined, the generated interference provides the direct, label-free, and real-time signal directly proportional to the analyte concentration.

Referring to FIG. 2E, a ring resonator transducer includes circular waveguides that generate whispering-gallery modes (WGM) upon light coupling to the loop, enhancing the evanescent field intensity. Detection is performed by interrogating the WGM spectral resonance position.

Other sensors that can be incorporated into a specimen container or in probe form include, but are not limited to those described in Cui, F., and Zhou, H. S. (2020) Diagnostic methods and potential portable biosensors for coronavirus disease 2019. Biosens. Bioelectron. 165, 112349; Qiu, G., Gai, Z., Tao, Y., Schmitt, J., Kullak-Ublick, G. A., and Wang, J. (2020) Dual-Functional Plasmonic Photothermal Biosensors for Highly Accurate Severe Acute Respiratory Syndrome Coronavirus 2 Detection. ACS Nano 14, 5268; and Seo, G., Lee, G., Kim, M. J., Baek, S. H., Choi, M., Ku, K. B., Lee, C. S., Jun, S., Park, D., Kim, H. G., Kim, S. J., Lee, J. O., Kim, B., T., Park, E. C., and Kim, S. I. (2020) Rapid Detection of COVID-19 1520 Causative Virus (SARS-CoV-2) in Human Nasopharyngeal Swab Specimens Using Field-Effect Transistor-Based Biosensor. ACS Nano 14 (4), 5135-5142, each of which are incorporated by reference herein.

As mentioned above, in vivo sample capture and testing devices can include a primary receptacle for receiving a fluid sample; and a sensor for label-free detection of a virus, the sensor being disposed at one side of the primary receptacle and configured to be coupled to a computing system. For supporting certain in vivo applications, the device can further include a needle. The primary receptacle can be disposed within a shaft of the needle (e.g., a lumen in the shaft). A probe of the sensor can also be disposed in a lumen of the shaft of the needle. For example, a probe containing one or more optical fibers and a coated optical element for BLI can be disposed in the lumen of the shaft of the needle.

The signal for the sensor can be amplified by including certain nanoparticles as a secondary reagent in the receptacle. Indeed, a primary receptacle can be predoped with the secondary reagent in advance of sample capture. The secondary reagent may include a combination of materials, for example an antibody or other moiety that binds to the analyte of interest. To increase the signal, the antibody or other binding moiety can be conjugated to a nanoparticle. Examples of potential nanoparticles include, but are not limited to, carbon-based nanoparticles, ceramic nanoparticles, quantum dots, silica nanoparticles, micelles, dendrimers, liposomes, metal nanoparticles, semiconductor nanoparticles, polymeric nanoparticles including nanospheres and nanocapsules, lipid-based nanoparticles, carbon nanotubes (CNTs) and fullerenes. In an example implementation, the nanoparticles can be gold nanoparticles. In some cases, further amplification can be achieved using heparin sulfate as a secondary binding agent (i.e., for the binding moiety). In some cases, further amplification can be achieved using spike antibody decorated nanoparticles or any other antibody to an antigen on the virus.

In some cases, for the amplification step it can be possible to increase sensitivity with nanoparticle conjugated secondary antibody. In one implementation, a fluid containing the nanoparticle conjugated secondary antibody can be provided. A patient can swish the fluid in their mouth prior to inserting a probe or the fluid can be infused in other locations prior to insertion of the probe/in vivo system configuration. In some cases of the in vivo system, the fluid containing the nanoparticle conjugated secondary antibody can be disposed in the receptacle (e.g., as a reaction reservoir within the probe). For ex vivo, the fluid can already be in the receptacle prior to sample collection or added after the fact.

The following Examples are provided by way of illustration and not by way of limitation.

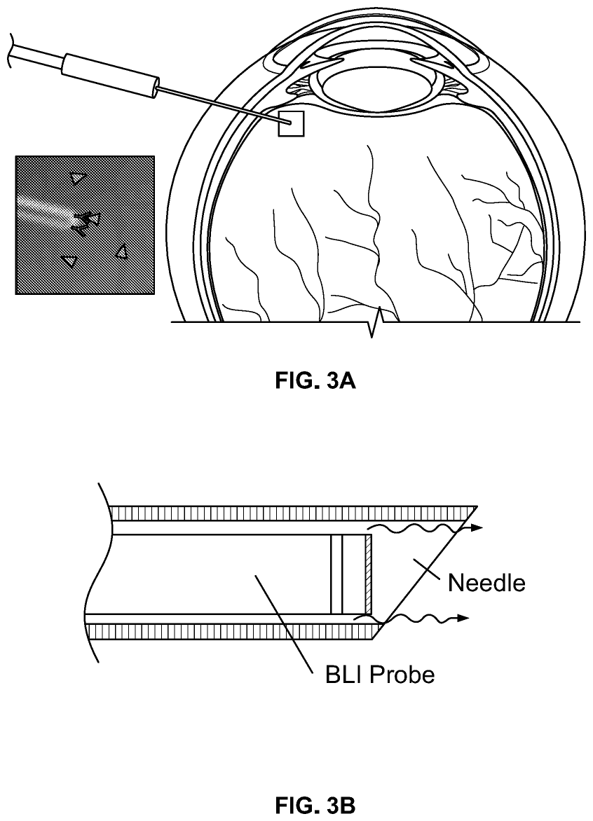

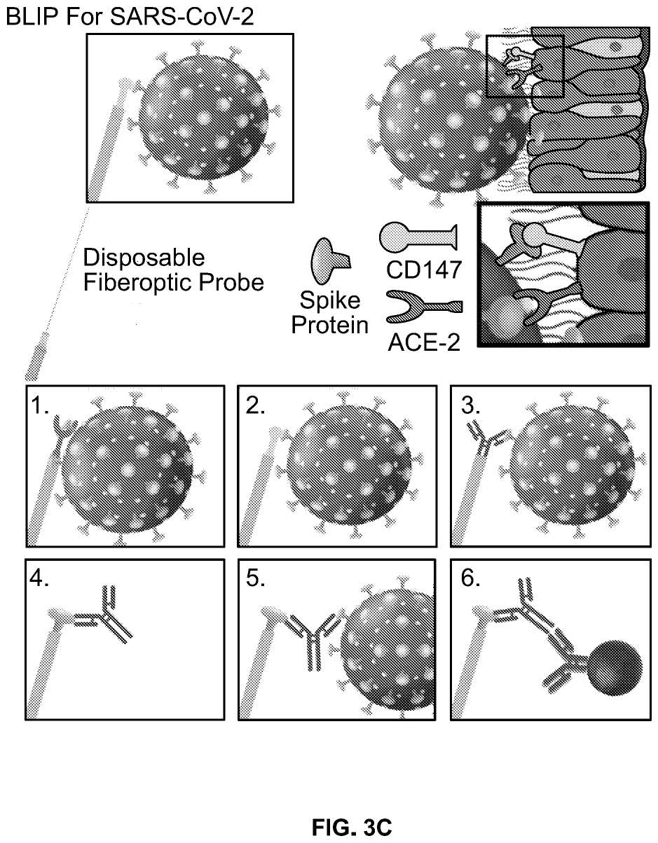

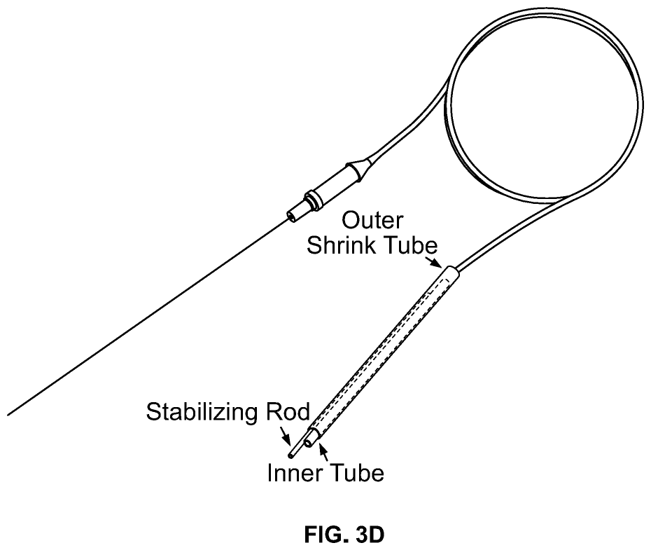

FIG. 3A depicts an example embodiment of a BLI probe. A means of sensitive in vivo detection of analytes is advantageous from a diagnostic and therapeutic standpoint. This is particularly true of difficult to access organs or fluids of low overall volume as well as of relatively lower concentrations. The eye is an example of such an organ. A BLI probe may enable the detection of various fluids in the eye such as aqueous humor, vitreous humor, suprachoroidal fluid and provide in vivo analysis of other bodily fluids including cerebrospinal fluid (CSF), synovial fluid, urine, saliva, pleural fluid, and other compatible body fluids. The application of such a technology is potentially far-reaching for the measurement of analytes in vivo including the detection of protein, RNA, DNA, therapeutics, and infectious agents.

In FIG. 3A, a BLI probe is shown inserted in a human eye; however, the probe is also suitable for other in vivo applications (see, e.g., Examples). The probe has an elongated rod-like shape. An optical element or lens is positioned at the distal end of the probe, and an outer surface of the optical element is coated with a first component of an analyte pair (e.g., an anti-analyte or ligand). The coating can be formed from any substance suitable for binding to the corresponding substance that is to be detected. Examples of binding components for SARS-CoV-2 are described with respect to FIG. 3C.

Examples of other ligand and analyte pairs include but are not limited to complementary nucleic acid pairs, antigen-antibody pairs, and receptor-receptor binding agents. Here, a robust and low-profile BLIP is utilized for measuring fluids of the eye including tears, aqueous humor, vitreous humor, subretinal fluid when present, and suprachoroidal fluid. For intraocular structures, said device involves a variety of delivery systems in order to enter the appropriate ocular space. For such applications, the BLIP is a low profile, mechanically stable fiber optic probe and optical coupler assembly for sensitive BLI.

In some embodiments, the BLI probe includes a plurality of optical fiber pairs, which can be used with a single optical element or with multiple optical elements coated with different analyte detectors. This allows the collection of multiple types of analytes. For detecting multiple analytes, the layer of analyte-binding molecules can be composed of an array of discrete analyte-binding regions. The regions are effective to bind different analytes. The optical fiber includes a plurality of individual fibers, each aligned with one of the regions, the detector includes a plurality of detection zones, and the optical coupling functions to couple each of the plurality of fibers with one of the zones. The analyte-binding molecules in the assembly can be, for example, an anti-species antibody molecules, for use in screening hybridoma libraries for the presence of secreted antibody; antigen molecules, for use in detecting the presence of antibodies specific against that antigen; protein molecules, for use in detecting the presence of a binding partner for that protein; protein molecules, for use in detecting the presence of multiple binding species capable of forming a multi-protein complex with the protein; or single stranded nucleic acid molecules, for detecting the presence of nucleic acid binding molecules.

Alternately, the probe can be provided as a plurality of single-analyte probes, which can be inserted serially or simultaneously into an incision, cavity, fluid, etc.

The optical element and optic fibers are captured in an outer housing or sheath. The sheath forms a smooth outer profile with the optical element in order to avoid injury to the surrounding tissue upon insertion, as well as providing mechanical rigidity. Additionally or alternately, the probe can be designed such that the probe is insertable through endoscopes, catheters, intrathecal probes, dialysis catheters, vitrectomy ports and needles, or other indwelling or procedurally inserted medical device. In one example embodiment, the BLI probe fits through 20 gauge to 30 gauge needles and ports.

FIG. 3B depicts an embodiment of an in vivo BLI probe within a needle shaft. The portion of the probe with the coated optical element and optical fibers can be disposed in the lumen. Optionally, the BLI system have one or more optical connections/couplings between the control portion and the probe tip. This allows the contaminated portion to be separated from the control portion, which can help minimize the consumable/disposable portion of the probe. In addition to housing the probe, the needle can optionally be configured to deliver a treatment to the detection site (indicated by arrows).

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

The term “effective amount” or “therapeutically effective amount” refers to an amount sufficient to effect beneficial or desirable biological and/or clinical results.

In other embodiments, the BLI probe itself has an integral needle tip for creating an insertion point into the bodily system. The needle tip is optionally beveled. The probe portion comprising the optical element is optionally extendable/retractable within the needle shaft.

Alternately, in other embodiments, the probe is used as a standalone device. The distal end of the probe has a smooth shape such that the probe does not cause damage or pain to the subject when inserted. This embodiment can be used, for example, in applications such as nasal or oral swabs.

The probe therefore has an overall width or diameter approximately the same size as a medical needle. That is, the cross-sectional extent of the probe is in the range of approximately 0.1 mm to 2.0 mm (0.004 inches to 0.08 inches). In some embodiments, a probe tip is sized to fit through standard clinical vitrectomy ports, including 20 gauge (0.9 mm diameter), 23 gauge (0.7 mm diameter), 25 gauge (0.5 mm diameter), and 27 gauge (0.4 mm diameter).

In other embodiments, the optical element is recessed from the tip of the probe. In these embodiments, the analyte can be drawn from the bodily system and measured in a “quasi-in vivo” manner. For example, the analyte can be drawn into the probe with a syringe or a wicking hydrostatic mechanism. The fluid is deposited into an incubation chamber that is at least partially bounded by the surface of the optical element that is coated with the ligand. The incubation chamber can be within the handle of the probe or beyond the handle of the probe. Further, there can be multiple optical elements disposed in the chamber, where each optical element is coated with a different type of binding substance. The optical elements can be disposed in series along a length of the chamber, and each optical element can independently measure the corresponding analyte. An advantage of a quasi-in vivo probe is that it is possible use a common device for multiple subjects. The disposable portion can be a microcuvette collection system within the handle that includes the optical element coated with the analyte detector and the tip that is inserted into the subject. A sterile bag can optionally be provided around the handle.

A BLI probe as disclosed herein is particularly useful for detecting diseases or infections by utilizing an antibody that binds to infectious agents for real time determination of the progression of the disease or infection. Optionally, the results of the detected analyte can be used to set a course of treatment. For example, if the BLI probe is used to measure the presence of a medication or other substance, the probe and/or the treatment delivery system can be left in situ until the desired outcome is achieved. Real-time feedback of the levels of a medication or other substance can lead to improved outcomes such as increased effectiveness and reduced number of treatments.

FIG. 3C illustrates interaction between a sensor probe and SARs-CoV-2. An understanding of point of care SARS-CoV-2 diagnostics requires an understanding of the molecular mechanisms behind SARS-CoV-2 infection. Many proteins involved in the entry of SARS-CoV-2 into cells as well as its replication have been identified, which are the targets of many therapeutics being developed. Both SARS-CoV-1 and SARS-CoV-2 have a spike glycoprotein that has been shown to interact with ACE-2. This interaction is currently believed to be necessary for viral penetration of host cell. In the case of SARS-CoV-2, currently it is believed that instead of ACE-2, CD147 can also act as the viral receptor. Prior to interaction with either receptor, the spike protein must first be cleaved by TMPRSS2. After this cleavage event, the virus binds to either ACE-2 or CD147 and is endocytosed.

Potential SARS-CoV-2 materials that can interface with sensor probes such as a FORTEBIO probe include, but are not limited to: 2019-nCoV (COVID-19) 3CL-Mpro Protein, His Tag, Human ACE2/ACEH Protein, Fc Tag, Biotinylated Human ACE2/ACEH Protein, His,Avitag™, Biotinylated Human ACE2/ACEH Protein, Fc,Avitag™, 2019-nCoV (COVID-19) Envelope protein, His Tag 2019-nCoV (COVID-19) Nucleocapsid protein, 2019-nCoV (COVID-19) S1 protein, His Tag, Biotinylated 2019-nCoV (COVID-19) S1 protein, His,Avitag™, SARS S1 protein, His Tag, 2019-nCoV (COVID-19) S protein RBD, 2019-nCoV (COVID-19) S protein RBD, Fc Tag, Biotinylated 2019-nCoV (COVID-19) S protein RBD, His,Avitag™, Biotinylated 2019-nCoV (COVID-19) S protein RBD, His,Avitag™ SARS S protein RBD, His Tag, 2019-nCoV (COVID-19) S protein Full Length, 2019-nCoV (COVID-19) S protein Full Length, Recombinant Coronavirus Envelope Protein (SARS-CoV ENV;1-76), Recombinant Coronavirus Matrix Protein (SARS-CoV; 182-216), Recombinant Coronavirus Membrane Protein (SARS-CoV, His tag), Recombinant Coronavirus Nucleoprotein (CoV-NP 229E), Recombinant Coronavirus Nucleoprotein (CoV-NP 229E), Recombinant Coronavirus Nucleoprotein (CoV-NP-NL63), Recombinant Coronavirus Nucleoprotein (SARS-CoV; 340-390), Recombinant Coronavirus Nucleoprotein (SARS-CoV; 340-390), Recombinant Coronavirus Nucleoprotein (SARS-CoV), Recombinant Coronavirus Nucleoprotein (SARS-CoV; 1-49), Recombinant Coronavirus Nucleoprotein (SARS-CoV; 1-49, 192-220), Recombinant Coronavirus Nucleoprotein (SARS-CoV; 340-390), Human CellExp™ Coronavirus Spike Protein (MERS-CoV S1; 18-725), Human CellExp™ Coronavirus Spike Protein (MERS-CoV S1; 18-725), Recombinant Coronavirus Spike Protein (MERS-CoV S1; 56-295), Recombinant Coronavirus Spike Protein (SARS-CoV S1; 12-53, 90-115, 171-203), Recombinant Coronavirus Spike Protein (SARS-CoV S1; His tag), Recombinant Coronavirus Spike Protein (SARS-CoV S2; 408-470, 540-573, His Tag), Recombinant Coronavirus Spike Protein (SARS-CoV S2; 408-470, 540-573), Recombinant Coronavirus Spike Protein (SARS-CoV S2; 408-470, 540-573), Recombinant Coronavirus Spike Protein (SARS-CoV S2), Recombinant Coronavirus Spike Protein (SARS-CoV S2), Recombinant Coronavirus Spike Protein (SARS-CoV S, His tag), Recombinant protein of human angiotensin I converting enzyme (peptidyl-dipeptidase A) 2 (ACE2).

One possible approach to label-free sensor-based COVID-19 testing would involve a BLI probe (e.g., BLIP) with an antibody to the Spike protein that could be placed in the mouth of a patient. As a society we have grown accustomed to oral temperature measurement. It could be transformative if a simple probe placed under the tongue or against the cheek of a patient could identify SARS-CoV-2 infection. Such a probe could also detect antibody against the virus. For example, a BLIP probe with a Spike protein could detect presence of antibody.

FIG. 3D illustrates a prototype configuration used for testing proof of principle for SARS-CoV-2 materials. To enable coupling to the FORTEBIO BLITZ, providing a BLI sensor, a fiber optic fusion splice sleeve was used to couple the fiber-optic cable to a tip and then an additional fiber optic fusion sleeve was used to attach an additional FORTEBIO tip at the distal end. In an example embodiment, the fiber optic cable would be continuous and would attach directly to the BLITZ.

FIGS. 4A and 4B show an example point of care sample capture and testing device. Referring to FIG. 4A, samples of saliva can be obtained through use of a specimen container 402 with removable specimen funnel 404. The specimen container 402 includes a primary receptacle for receiving a fluid sample. Once the fluid sample, such as saliva, is received in the specimen container 402 via the specimen funnel 404, the specimen funnel 404 can be removed (406) and a specimen cap 408 can be attached (410) to the opening of the specimen container 402. The specimen cap 408 can have a foil lid portion or a valve for receiving a sensor tip.

Referring to FIG. 4B, a sample capture and testing device includes a specimen container holder 412, a plurality of sensor tips 414, a sensor chamber 416 configured to hold the plurality of sensor tips 414, a sensor tip advancer 418, a coupler 420, and a bar 422.

The sensor tip advancer 418 causes a new sensor tip to be fed to the coupler 420. The sensor chamber 416 can be in the form of a channel with openings through which a sensor tip extends. The sensor tip advancer 418 can be a spring or elastic member or other material that can create a tension that pushes the sensor tips 414 towards the front of the sensor chamber and to the coupler 420.

The coupler 420 has a housing that receives a sensor tip advanced from the chamber 416 by the sensor tip advancer 418. The sensor tip advancer automatically advances the sensor tip when the housing is empty (e.g., due to the force maintained by advancer 418). In some cases, the sensor tips are permitted to advance in response to an activating motion, such as by a user depressing a lever/handle (which may be attached to or part of the bar 422) causing a release/opening for the sensor tip advancer to move the sensor tips forward).

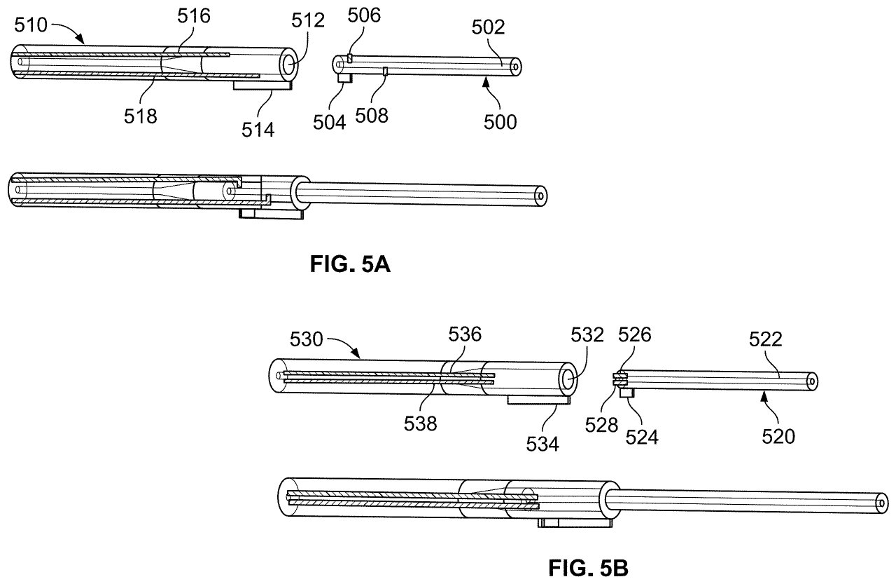

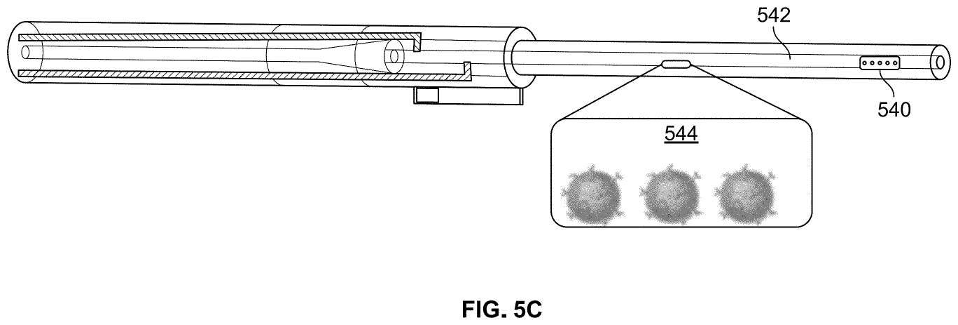

The bar 422 can be depressed or otherwise have pressure asserted thereon, causing the sensor tip located in the coupler 420 to be pushed into the cap 408 of a specimen container 402 located in the container holder 412 of the system. In some cases, the sensor tip is pushed through the opening of the chamber to penetrate or enter through an opening of the specimen cap. In some cases, the sensor tip is released from the chamber before penetrating or entering the specimen cap. The application of pressure to the bar also couples the remaining components of the sensor (not shown) to the sensor tip. For example, a connector can be disposed in the bar such that when the bar is depressed, the connector releasably couples the sensor components to the sensor tip. In some cases, the sensor components that may be in the coupler 420 and the sensor tip 414 can be configured similarly to the in vivo tip and components shown in FIGS. 5A-5C.

In some cases, a disposal container 424 can be included that is configured to capture the sensor tip after use. The container holder can be coupled to a hinge 426 and elastic member 428 that causes the specimen container to be ejected or released from the container holder into the disposal container when the pressure at the bar is released.

In some cases, instead of holding a single specimen container, the specimen container holder 412 can hold a plurality of specimen containers and the coupler 420 supports coupling a corresponding plurality of sensor tips to the plurality of specimen containers. Multiple tips can thus be exchanged out simultaneously based on the cartridge configuration.

In some cases, the system can enable reuse of sensor tips by including at least one regeneration station. Regeneration of the probe may occur through the use of a dissociation buffer that causes the dissociation of the diagnostic binding pair. This dissociation may be accomplished by altering pH, temperature, tonicity, magnetization, or other local property. By removing the diagnostic binding pair, the system is capable of measuring concentration of an analyte in an additional sample.

FIG. 4C shows an implementation of a sample capture and testing device with reusable sensor tips. Referring to FIG. 4C, one or more sensor tips can be included in the device and sequentially advanced to stations within the system. The stations include sample, buffer(s), and rinse. In the example shown in FIG. 4C, the stations include a sample station 430, which can contain a container holder (e.g., container holder 412), a blank buffer station 432, a rinse station 434, a regeneration buffer station 436, and a final rinse station 438. The stations can be disposed in order in a circular arrangement. In such a case, a carousel 440 can hold one or more sensor tips 442, for example, by connectors, grips, or other suitable fasteners. The carousel may be configured as a mobile or a wheel as examples. When a bar such as bar 422 is actuated and released, the carousel 440 can rotate to a next position. For example, the carousel 440 can rotate the plurality of sensor tips from the specimen container holder sequentially to a buffer station of the at least one buffer station to a rinse station of the at least one rinse station and return to the specimen container holder as the bar is applied in subsequent operations. In some cases, one sensor tip is provided on the carousel. In some cases, an equal number of sensor tips to stations is provided. In some cases, more sensor tips are included, resulting in some sensor tips not actively in used or being prepared. In some cases, multiple sensor tips may be able to be dipped into one or more of the stations.

In some cases, a plate washer (multiwash microplate washer) configuration may be used.

In one embodiment, a secondary antibody conjugated to ferromagnetic nanoparticles can be recovered by a magnetic field. In the setting of magnetic stripping, the magnetic nanoparticle could be dissociated from the diagnostic probe but still have analyte bound. This in turn would require the addition of dissociation buffer to cause analyte dissociation from magnetic nanoparticle. The use of variable electromagnetic magnet may enable binding and release of secondary nanoparticles. Such an approach would enable regeneration of both diagnostic probe and secondary magnetic nanoparticle. FIG. 4D shows a plot of concentration over time, which shows that recalibration can be performed after regeneration to build a signal for baseline prior to sample measurement when reusing a tip.

Recalibration can be performed after regeneration to build a signal for baseline prior to sample measurement when reusing a tip.

In some cases, the computing system can include instructions that direct the carousel to advance to next position only when readings at certain stations have appropriate values.

In some cases, the computing system can automate bath changeout for regeneration (and may include built-in signal zeroing). For example, the system can take a reading in the dissociation bath and the tip remains there until the value is reduced to a certain set point or until a plateau is detected and the system appreciates no further dissociation. If the signal does not drop to an appropriate level in the dissociation bath, the system can require a replacement of bath and tips simultaneously.

In order to judge dissociation of analyte from the biosensor, the biosensor may be in an active channel measuring signal while in a dissociation buffer. When signal reduction plateaus, the diagnostic tip can be determined as regenerated and moved to a channel for measurement of a new sample. The new baseline signal can be incorporated into future measurements so the delta between signal at the end of regeneration at each cycle and new sample measurement can increase precision of measurement. This similarly enables a quality control mechanism to ensure diagnostic sensor and regeneration buffer are effective. Regeneration can also be accomplished based upon empiric determination of the maximal necessary time to enable full dissociation of analyte from biosensor and building into a system the necessary dwell time. Following dissociation, prior to new sample measurement a baseline signal can be obtained.

In order to ensure sufficient volume is present in regeneration bath and other necessary fluids in the system, a current with voltage meter, light source with light meter, pH meter or any other system to determine absence of fluid, pH of fluid, turbidity of fluid or any other feature indicative of suboptimal bath may be incorporated. This in turn can create a fluid exchange not unlike a multiwell plate washer or alternatively may signal to the end user to manually change the fluid. In one implementation, the readings for analyzing presence of fluid and quality of fluid in the buffer baths involve a light source and a light sensor. In another implementation, a current is applied. When fluid is empty the charge can no longer be transmitted and the system requests refill. When the quality of the solution changes this may also impact current. In some implementations, pH and temperature can be monitored. Change in pH or turbidity of solution may also be an indication that the regeneration buffer or bath needs to be replaced.

In some cases, the testing for replacement of tip and/or solutions can be based on tip type and sample types.

In some cases, a coupled computing system can include instructions that direct the system (e.g., as operating the carousel) to serially measure the same sample with different tips which could act as an internal control. In some cases, internal variation between individual measurements in the same sample can cause the system to indicate need for or cause replacement of sensor tips or buffers.

In addition to liquid handling for buffer exchange, a means of biosensor tip exchange can be incorporated. The replacement of biosensors may be empirically determined a priori for maximum assays. The need for biosensor tip replacement may also be determined in real time due to inability to achieve adequate signal in the active channel or absence of signal in the regeneration or wash buffer channel. The system may include a means of single biosensor exchange or alternatively may contain a cartridge containing multiple tips, for example in a carousel array.

In addition to a biosensor tip physically moving between buffer and sample, a system in which fluid is exchanged in a single reaction vessel can be incorporated. In this configuration, instead of going from bath to bath, the biosensor tip remains in one location and the fluid within that location has inflow and outflow. A similar feedback loop as described above, from in which the regeneration bath is not drained until the signal drops to a certain level and if it does not drop to that level a new tip is required can be incorporated into the system.

FIGS. 5A and 5B illustrate various multiplex options and FIG. 5C illustrates a multiplex option with secondary antibody/antibody nanoparticle in a capillary reservoir. These implementations are suitable for testing of multiple biological components in a single sample. For example, both SARS-CoV-2 and the flu virus can be tested. Referring to FIG. 5A, in one multiplex configuration, a sensor tip 500 can include a capillary tube 502 (which may be considered a primary receptacle), an alignment notch 504, and at least a first probe tip 506 and a second probe tip 508. In this implementation, the probe tips 506, 508 are perpendicular to the capillary tube 502. The sensor tip 500 couples to a sensor component 510, which includes an opening 512 that receives the sensor tip 500 and an alignment slot 514 that receives the alignment notch 504 to align the first probe tip 506 and the second probe tip 508 with a corresponding first fiberoptic line 516 and second fiberoptic line 518 of the sensor component 510. In some cases, the sensor tip 500 has an alignment slot and the sensor component 510 has an alignment notch.

Referring to FIG. 5B, in another multiplex configuration, a sensor tip 520 can include a capillary tube 522 (which may be considered a primary receptacle), an alignment notch 524, and at least a first probe tip 526 and a second probe tip 528. In this implementation, the probe tips 526, 528 are disposed at a proximal end of the sensor tip 520 along a same axis as the capillary tube 522. As with the implementation of FIG. 5A, the sensor tip 520 couples to a sensor component 530, which includes an opening 532 that receives the sensor tip 520 and an alignment slot 534 that receives the alignment notch 524 to align the first probe tip 526 and the second probe tip 528 with a corresponding first fiberoptic line 536 and second fiberoptic line 538 of the sensor component 530. In some cases, the sensor tip 520 has an alignment slot and the sensor component 530 has an alignment notch.

Referring to FIG. 5C, a filter or valve 540 can be disposed at one end of the primary receptacle of the capillary tube 542. In some cases, lyophilized antibody or antibody decorated nanoparticles 544 can be disposed directly in the tube 542 or in a lumen of the tube 542.

FIGS. 6A and 6B illustrate sensor tips integrated in a sample capture tube. Referring to FIG. 6A, a sensor tip, such as a BLI tip, can be incorporated into the base of the capture tube container. In some cases, a secondary antibody or nanoparticle can be lyophilized inside the container (in a primary receptacle or in a secondary receptacle). The patient spits in the container and seals the container with a cap. The container is then placed in a reading unit such as shown in FIG. 6B, which couples with the base of a specimen container (e.g., with a notch and slot) for immediate readout. The reading unit can include a specimen container holder with coupler configured to couple the sensor tips to a remaining component of the sensor. Through use of such a base, multiple specimens can be measured in parallel.

In some cases, the capture tube can be a cryovial, which can be placed on the base and, after performing measurements, stored in cryostorage.

In both the in vivo and the point of care implementations, the sensors can be incorporated into the base or wall of the collection container. This helps avoid having to advance a delicate probe through a valve or seal (e.g., on the cap). There can be numerous sensor tips in the container that can couple with a receptacle in a sensing unit.

FIG. 6C illustrates a serum separator configuration. Here, instead of saliva, blood can be collected and a filter 650 such as a gel can be included in the container to facilitate separation for testing. In such a case, the testing system can include a centrifuge component (not shown). For example, the centrifuge component can include a chamber configured to hold the primary receptacle in a position for rotation about a fixed axis; and a motor configured to rotate the chamber with centrifugal force such that the fluid sample enters the primary receptacle through the filter and proximate the one side at which the sensor is disposed. This enables an amount of post processing to be performed after collection of the sample, prior to testing. As mentioned, the fluid sample (e.g., separated serum) enters the primary receptacle through the filter to become proximate the sensor so that the sensor can detect analytes (or other components of interest) in the fluid.

Advantageously, by decorating any probe or container with the desired probes, there is no need to extract the sample from the container in order to perform testing. This can improve safety of those handling the potentially infectious agents.

As mentioned above, it is possible to provide POC or in vivo analysis, when an end user dips a probe in a sample or for the probe to be inserted into the mouth or another location of the patient. In the case of probe being added to the sample, a lid to the sample with valve or pierceable material such as foil may enable the probe to be inserted into the sample container. In the event of a delicate biosensor, the probe may have a protective sheath that prevents damage to the biosensor as it passes through the container. Such a sheathed-based approach may also protect a delicate biosensor to be inserted in vivo. Through capillary like action, the sample can then be drawn within the sheath for exposure to the biosensor. In addition to capillary action, a manual or automated suction can pull the solution to be analyzed into the reaction chamber. In certain implementations, the fluid may represent otherwise unmodified body fluids such as saliva, urine, or cerebrospinal fluid. In an alternate implementation, a diagnostic fluid can be infused in sample location to mix with local analytes. Examples of such a system include an oral rinse that can modify the local fluid and may also add secondary antibody or other reagents. A similar rinse may also be beneficial in respiratory locations such as a nasal or bronchiole lavage. To achieve infusion of this rinse, in addition to an aspiration channel the system may include an infusion channel.

The local pH, temperature, protein content, viscosity or other physical feature may modify biosensor binding and thus signal. The system may contain a thermometer, pH meter or other sensor that enables real time adjustment for individual sample fluid characteristics. Alternatively, known standards can be established for each fluid type. Due to changes found with age, sex, species and other patient specific characteristics, a baseline normative database may be established that takes into account binding pair as well as the baseline characteristics of the environment in which the analyte is present. In certain implementations a surrogate analyte may act to normalize the total signal of the analyte of interest. As an example, a collagen binding protein on the sensor tip may act to normalize the signal of another analyte measured by a separate tip in the vitreous humor of the eye. The ability to a priori empirically normalize or actively normalize the sample would be incorporated in the system described as the ability to generate a standard curve is precluded for this scenario.

Results

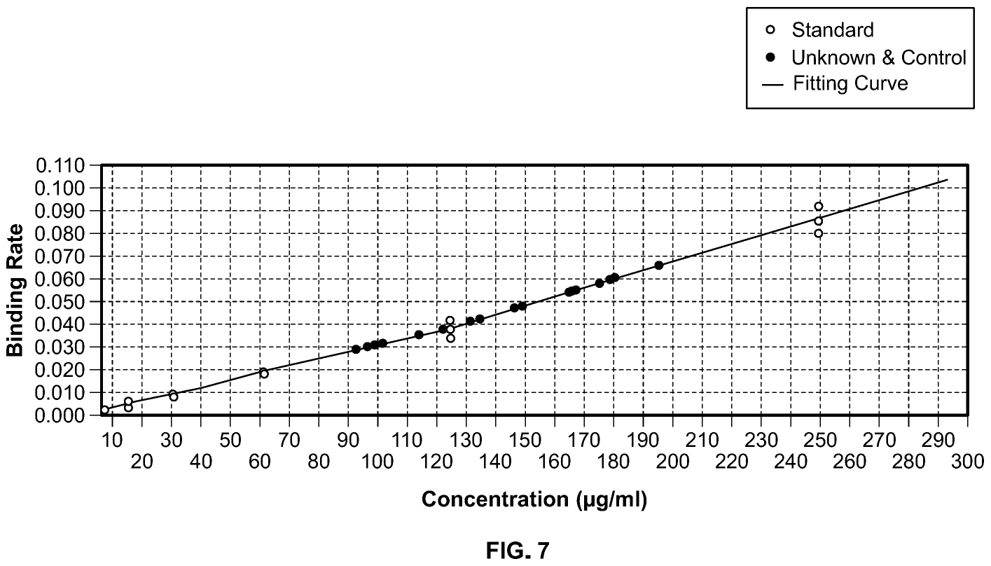

As a proof of principle experiment for the ability of BLI to detect proteins in saliva, ranibizumab was added to saliva. A Forte octet unit with protein L tips was utilized to detect ranibizumab in saliva at various concentrations. FIG. 7 shows a plot of concentration to binding rate. As can be seen, the probe detected ranibizumab along a linear fitting curve.

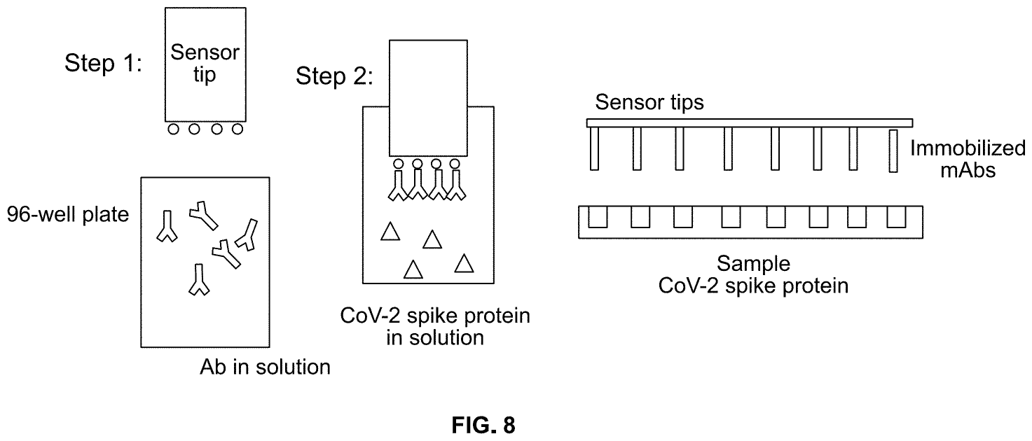

FIG. 8 shows a representation of biosensor capture for sensor testing of AHC (anti-human Fc), ProA (Protein A), and AR2G (amine coupling) with respect to capture of Cov2 Abs. The captured antibodies at 20 u/ML included Cat_Ab82_AAA(3369) Aliq. Nov. 16, 2017 (control); Ab712199 LS/293i/Citrate; and Ab026065 LS/293i/Citrate. The antigens screened at 50 ug/ML included nCoV-1 nCoV2/293F/post-SEC/nCoV buffer/078VS. Referring to Figure X, for the assay, in step 1, mAbs are directly captured onto sensor tips; and, in step 2, mAb captured sensor is then submerged into the proteins to detect binding response. These tests were carried out to determine the stability of capture by testing the spike protein at a single conc and compared the spike protein binding in two cycles of testing. It should be noted that these data do not provide any measure of antigen sensitivity.

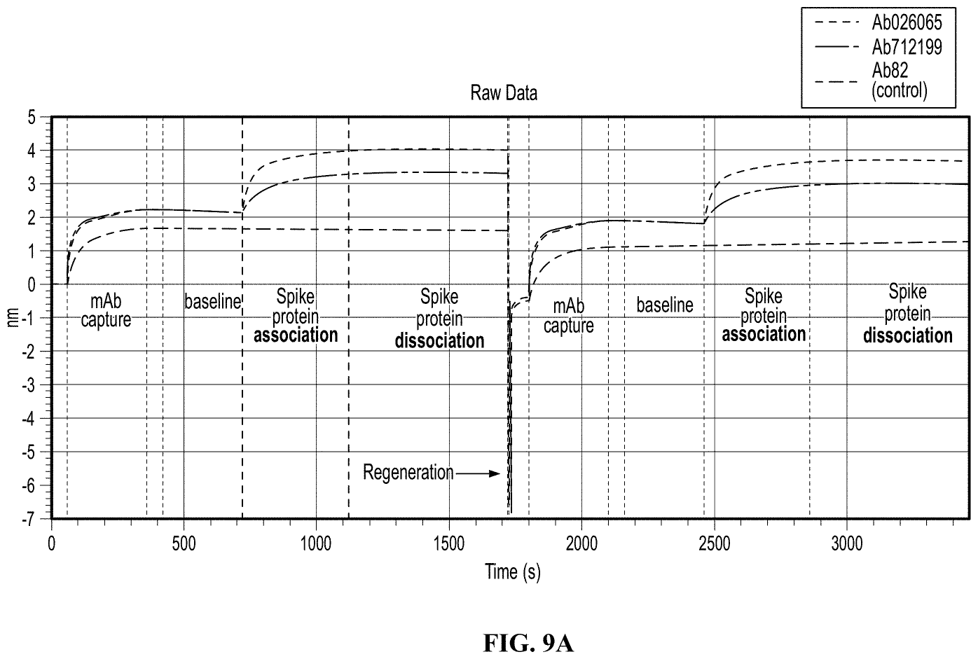

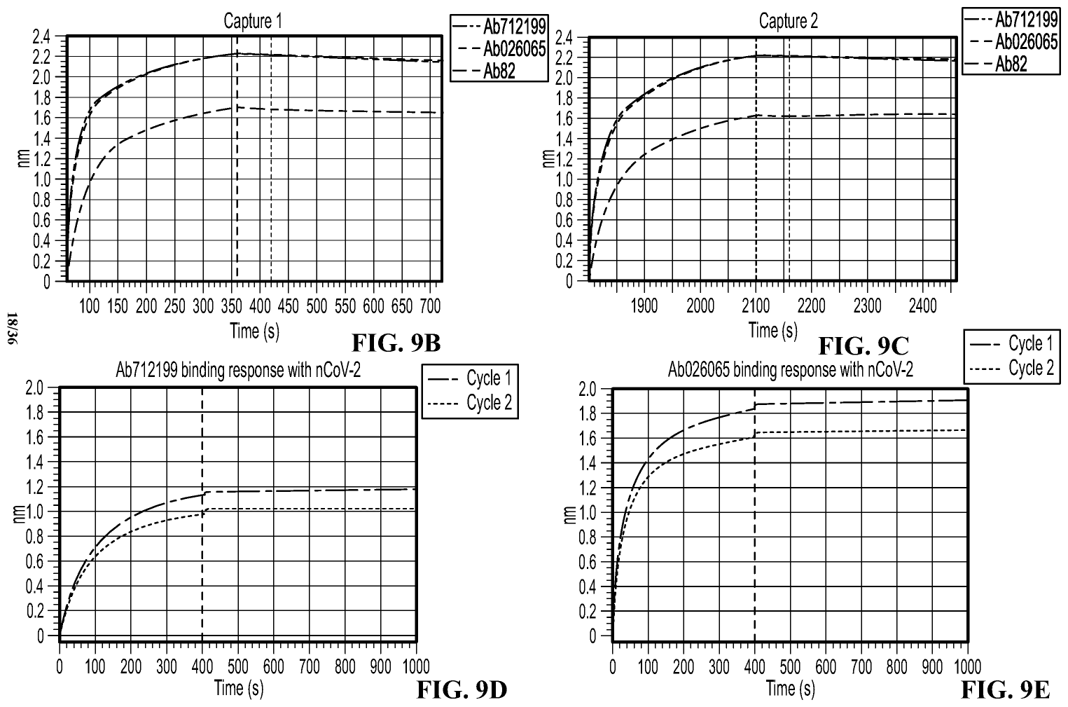

FIGS. 9A-9E show testing with BLI-AHC sensors. For the anti-human FC (AHC) sensors, antibody (Ab) capture was stable for both antibodies and remained stable after regeneration. In addition, bindings of Cov-2 Abs to spike protein were similar in two replicates.

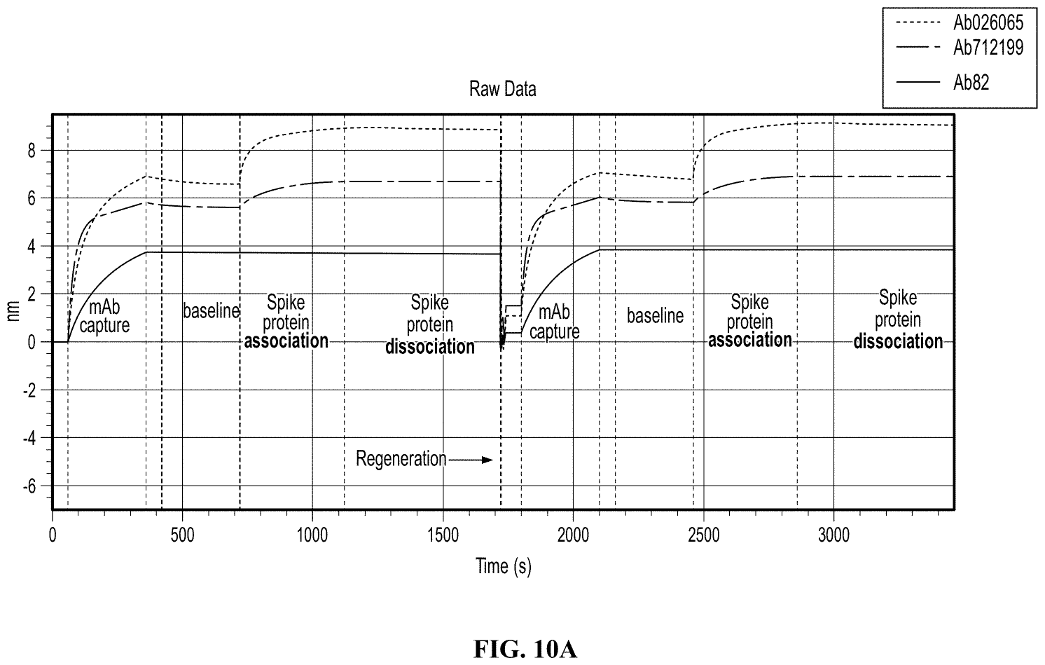

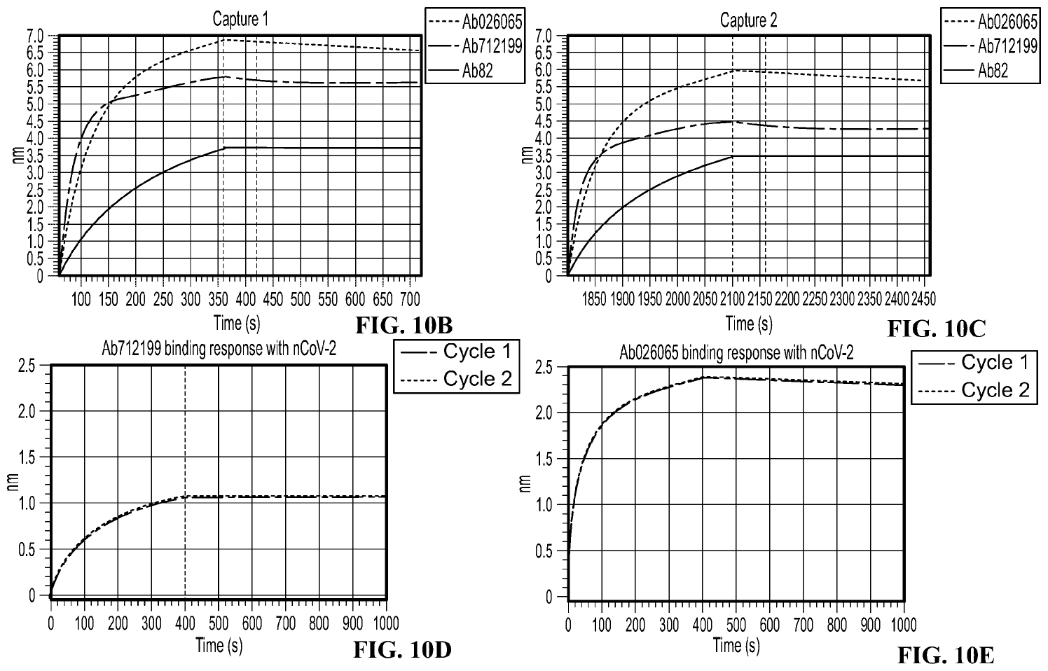

FIGS. 10A-10E show testing with BLI-ProA sensors. For the protein A (ProA) sensors, a much higher level of each Ab was captured and variation in Ab capture was observed in two cycles. Ab conc used in the capture may need to be optimized. Binding responses of Cov-2 Abs to spike protein were identical and remained unchanged in two cycles of testing.

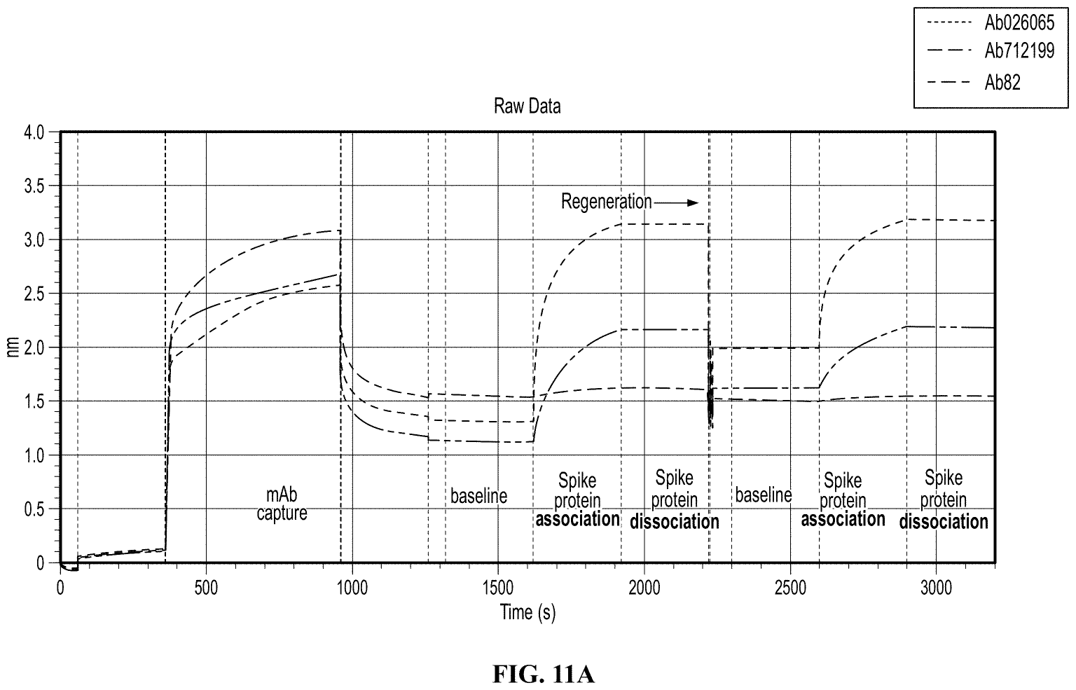

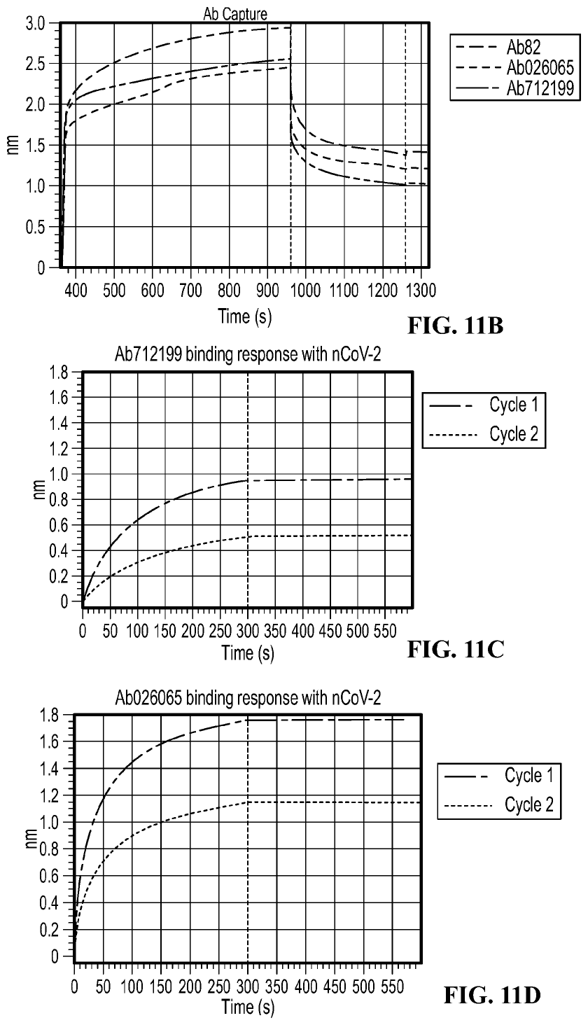

FIGS. 11A-11D show testing with BLI-AR2G sensors. For the Amine Coupling (AR2G) sensors, binding responses of Cov2 Abs were reduced after regeneration. Optimization of regeneration condition may improve reproducibility.

As can be seen from the plots, both AHC and ProA sensors gave stable capture and similar binding responses in two cycles of testing. These two sensors can be used without further optimization, except that on the ProA a lower Ab conc is used for capture on the sensor.

The AR2G also gave stable and comparable capture but the spike protein was sensitive to the regeneration buffer. Accordingly, the regeneration condition should be optimized. With both AHC and ProA sensors, the Ab capture is done in a single step and that makes them relatively simpler to use.

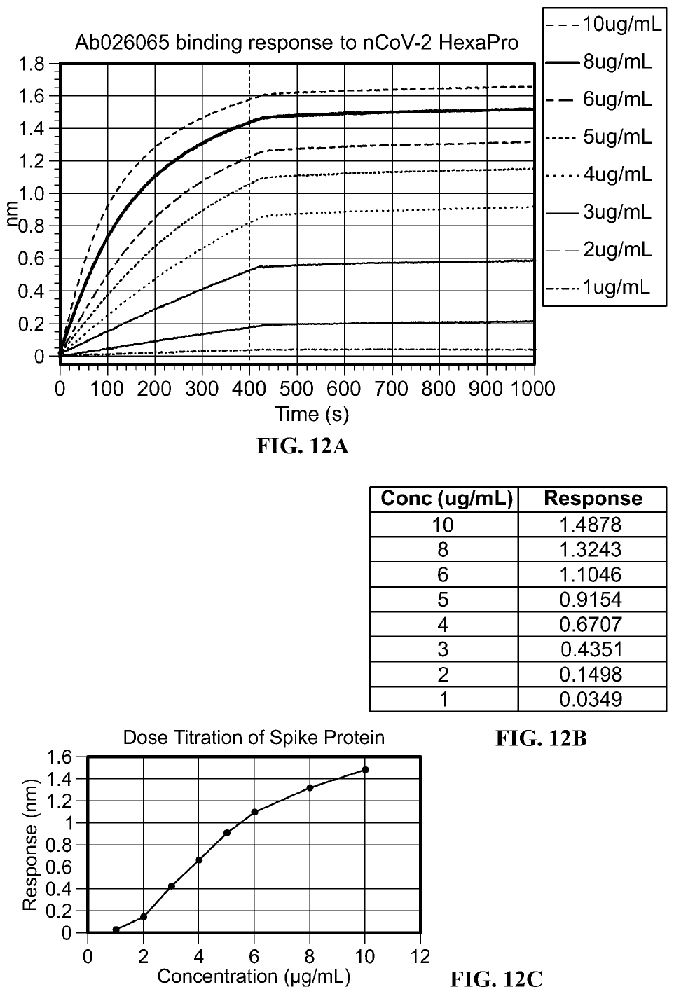

FIGS. 12A-12C show titration data for BLI-AHC Ab026065 binding response to nCoV-2 HexaPro, where FIG. 12A shows a plot of binding time to response over various concentrations outlined in the table shown in FIG. 12B, and FIG. 12C shows a plot of concentration to response for dose titration of spike protein. As can be seen from these data, it is possible to directly detect about lug/ml.

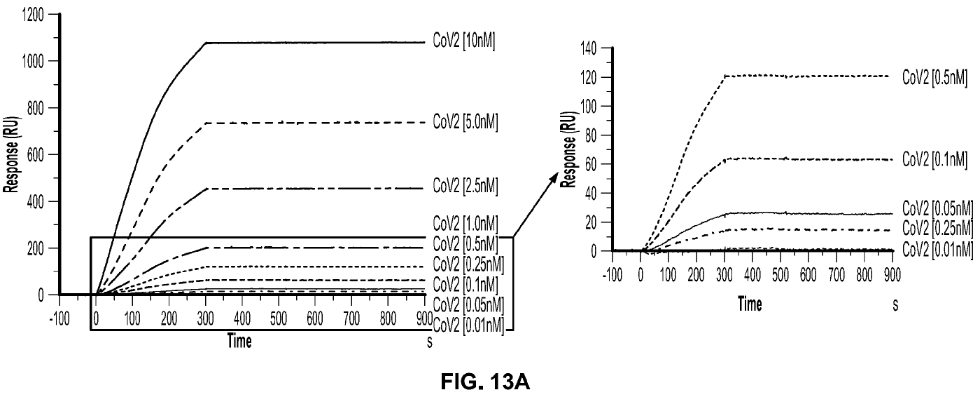

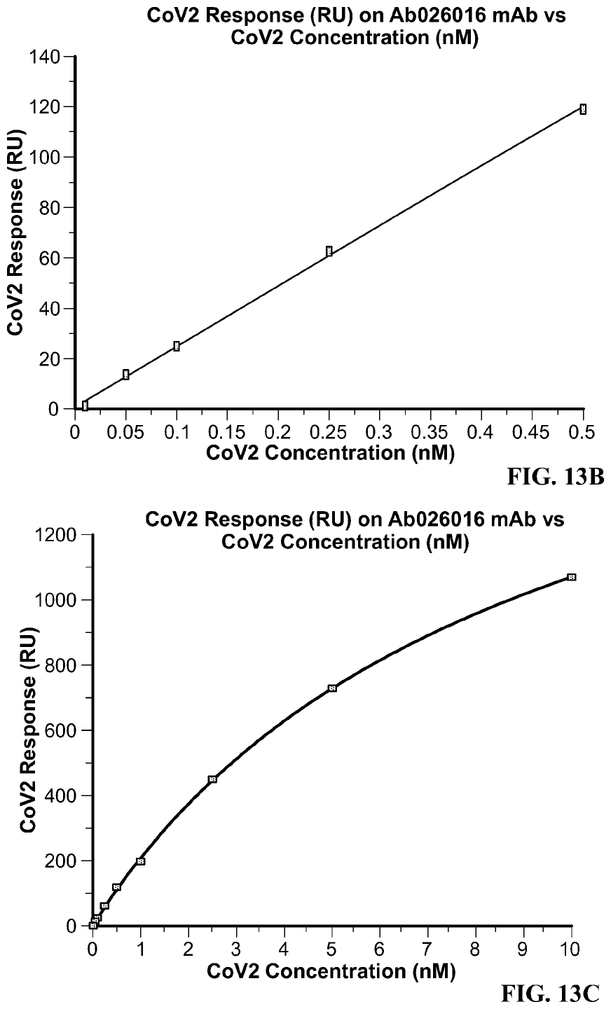

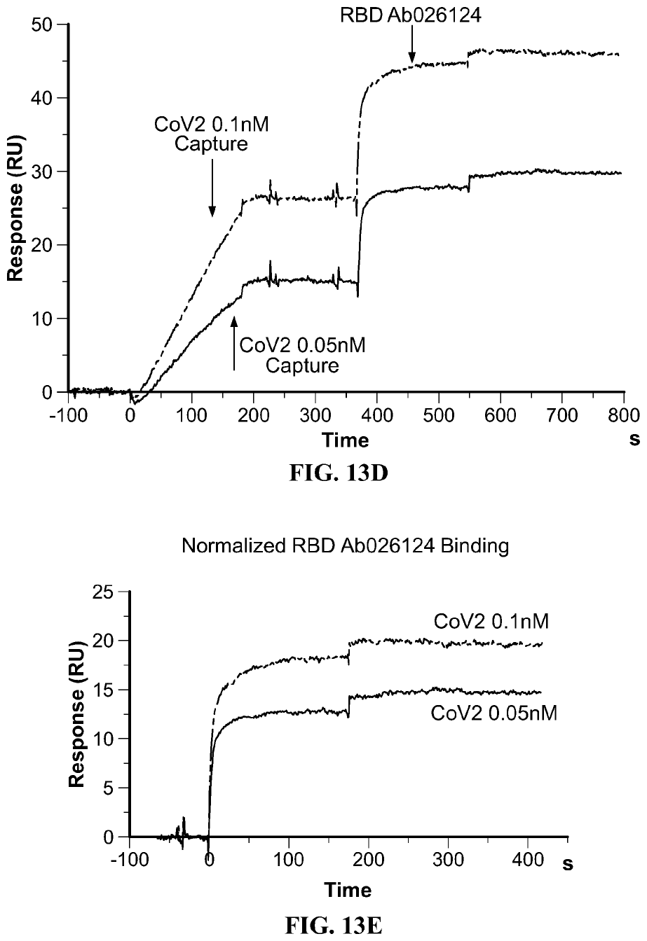

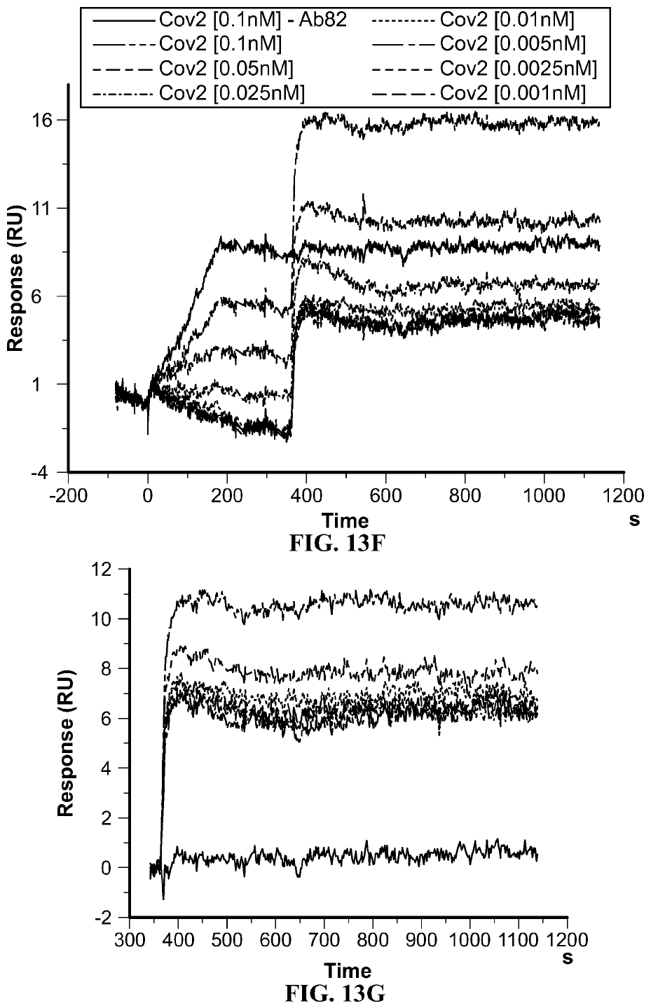

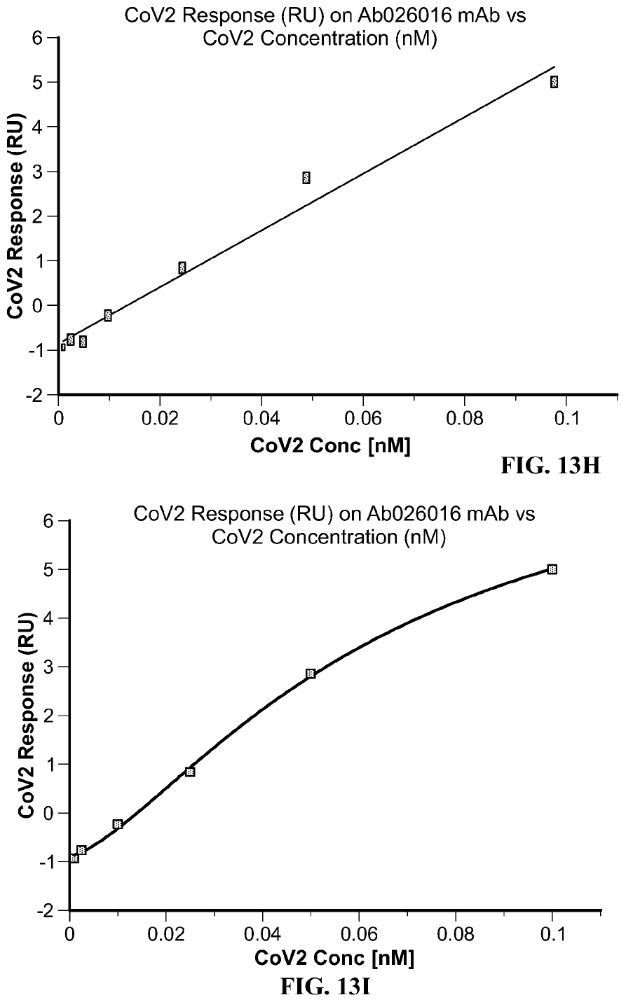

In another study, a pair of high affinity receptor-binding domain (RBD) and n-terminal domain (NTD) Abs were used in a direct binding to CoV2 spike protein and a sandwich assay. FIGS. 13A-13I show plots for a titration dataset. FIGS. 13A-13C show plots for nCoV-1 nCoV-2P on NTD Ab026016 mAb, where FIG. 13A shows a plot of protein binding time to response, FIG. 13B shows a linear regression from CoV2 Conc: 0.01-0.5 nM in a plot of CoV2 Response (RU) on Ab026016 mAb vs CoV2 Concentration (nM), and FIG. 13C shows a 4-parameter equation from CoV2 Conc: 0.01-10 nM in a plot of CoV2 Response (RU) on Ab026016 mAb vs CoV2 Concentration (nM).

FIGS. 13D-13I show plots for nCoV-1 nCoV-2P sandwich on NTD Ab026016 and RBD Ab026124 mAb, where FIG. 13D shows a plot of protein binding time to response and FIG. 13E shows normalized RBD Ab026124 binding for Cov2 concentrations of 0.05 nM and 0.1 nM. FIGS. 13F and 13G show plots of protein binding time to response and normalized RBD Ab026124 binding, respectively, for Cov2 concentrations of 0.1 nM—Ab82, 0.1 nM, 0.05 nM, 0.025 nM, 0.1 nM, 0.005 nM, 0.0025 nM, and 0.001 nM. FIG. 13H shows a linear regression from CoV2 Conc: 0.01-0.1 nM in a plot of CoV2 Response (RU) on Ab026016 mAb vs CoV2 Concentration (nM), and FIG. 13I shows a 4-parameter equation from CoV2 Conc: 0.001-0.1 nM in a plot of CoV2 Response (RU) on Ab026016 mAb vs CoV2 Concentration (nM).

Referring to FIGS. 13A-13I, as can be seen, the detection limit is around 25 pM (˜0.025 nM) and the sandwich assay amplified the signal response (but did not appear to significantly improve detection limit).

It is to be noted that the above-mentioned embodiments are merely exemplary and not intended to limit the scope of the invention. Such systems and devices may have many additional foreseen and unforeseen applications that will be apparent to a person of skill in the art. For example, certain of the disclosed approach may potentially enable in vivo analysis of other bodily fluids. Example fluids include cerebrospinal fluid (CSF), synovial fluid, pleural fluid, amniotic fluid, pericardial fluid, urine, saliva, seminal fluid, vaginal secretions, and other compatible body fluids. The application of such a technology would be potentially far-reaching for the measurement of analytes in vivo including the detection of protein, RNA, DNA, therapeutics, and infectious agents.

Advantageously, the devices and systems disclosed herein can potentially address current shortcomings of SARS-CoV-2 diagnostics. In order to effectively control SARS-CoV-2 a means of rapid, quantitative testing with high accuracy is needed. Due to the high percentage of asymptomatic individuals with SARS-CoV-2, a diagnostic system to reduce disease transmission must be amenable to universal rapid implementation. The described devices and systems can be used in screening settings to enable real-time diagnosis, leading to more effective self-isolation and reducing transmission in high risk groups. In many instances over the course of multiple days during which testing processing occurs, SARS-CoV-2 spreads rapidly amongst the high-risk population. The ability to immediately identify which persons had antibodies and potential immunity to the virus as well as those that had active virus would be invaluable in reducing spread during this diagnostic time delay.

It is well understood that rapid diagnostics are critical as well to inform local and national public health policies. Current SARS-CoV-2 testing strategies utilizing nasopharyngeal swabs that are then post-processed with real-time RT-PCR have proven problematic. Even in the instance of same day testing, persons must then be re-contacted. By using the described devices and systems it is possible to obtain immediate results at the point of care.

Point of care testing has become popular both in non-clinical and clinical settings, due to relatively low cost and rapid results. However, this has also led to a backlog of samples to test. In many instances it is not just the presence of a substance but the level of the substance that is critical. For example, to detect antibodies to SARS-CoV-2, a quantitative assessment of antibody levels against the virus could provide far greater information about the likelihood of immunity. In the same way, a point of care test that could provide a quantitative level of virus in various locations in the body would be invaluable for better understanding of disease pathogenesis and prognostication. For example, absence of virus in the lungs with a robust immune response may prompt modification of local or systemic medication modification away from aggressive anti-virals to potentially some form of local immunosuppression.

As mentioned above, the described systems and devices can be configured to detect SARS-CoV-2 virus or antibodies to the virus in saliva as an oral probe, in nasal mucous as a nasal probe, detection in tears as an ocular surface probe, detection in respiratory bronchiole, or in GI tract with fiber optic cable in endoscope working channel.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A sample capture and testing system, comprising:
a primary receptacle for receiving a fluid sample; and
a sensor for label-free detection of an analyte, the sensor being disposed at one side of the primary receptacle and configured to be coupled to a computing system.

2. The sample capture and testing system of claim 1, wherein the primary receptacle is predoped with a secondary reagent.

3. The sample capture and testing system of claim 2, wherein the secondary reagent comprises a binding moiety.

4. The sample capture and testing system of claim 3, wherein the binding moiety comprises heparin sulfate on gold nanoparticles.

5. The sample capture and testing system of claim 1, further comprising:
the computing system, wherein the computing system comprises a processor, a storage device, and instructions stored on the storage device that direct the computing system to:
obtain a baseline measurement for the sensor;
store the baseline measurement in the storage device;
obtain a sample measurement from the sensor;
compare the sample measurement with the baseline measurement; and
output an indication of presence or absence of the analyte to a display.

6. The sample capture and testing system of claim 5, wherein the baseline measurement is from historical captured data.

7. The sample capture and testing system of claim 5, wherein the baseline measurement is obtained from the sensor before obtaining the sample measurement from that sensor.

8. The sample capture and testing system of claim 1, further comprising a needle;
wherein the primary receptacle is disposed within a shaft of the needle, and wherein a probe of the sensor is disposed in a lumen of the shaft of the needle.

9. The sample capture and testing system of claim 1, further comprising:
a secondary receptacle for receiving the fluid sample, the secondary receptacle having a second sensor for label-free detection of a second analyte disposed at one side.

10. The sample capture and testing system of claim 9, further comprising a needle;
wherein the primary receptacle is disposed within a shaft of the needle,
wherein a probe of the sensor is disposed in a lumen of the shaft of the needle,
wherein the secondary receptacle is disposed within the shaft of the needle, and
wherein the second sensor is disposed in a second lumen of the shaft of the needle.

11. The sample capture and testing system of claim 9, wherein the analyte is a target analyte of SARS-CoV-2 and the second analyte is a target analyte of a flu virus.

12. The sample capture and testing system of claim 1, further comprising:
a second sensor for label-free detection of a second analyte disposed at a side of the primary receptacle.

13. The sample capture and testing system of claim 1, further comprising a filter at an opening of the primary receptacle that receives the fluid sample.

14. The sample capture and testing system of claim 13, further comprising:
a chamber configured to hold the primary receptacle in a position for rotation about a fixed axis; and
a motor configured to rotate the chamber with centrifugal force such that the fluid sample enters the primary receptacle through the filter and proximate the one side at which the sensor is disposed.

15. The sample capture and testing system of claim 13, wherein the filter comprises a gel for serum separation.

16. The sample capture and testing system of claim 1, wherein the sensor comprises a BLI probe.

17. The sample capture and testing system of claim 1, further comprising:
a specimen container, wherein the primary receptacle is disposed within the specimen container;
a specimen container holder with coupler configured to couple the sensor at the side of the primary receptacle to a remaining component of the sensor.

18. A sample capture and testing system, comprising:
a specimen container holder;
a plurality of sensor tips;
a coupler comprising a housing that receives a sensor tip of the plurality of sensor tips; and
a bar configured to couple the sensor tip to a remaining component of a sensor and computing system, the bar further configured to apply pressure to the sensor tip to push the sensor tip into the specimen container holder.

19. The sample capture and testing system of claim 18, further comprising:
a sensor chamber configured to hold the plurality of sensor tips to be fed to the coupler;
a sensor tip advancer that causes the sensor tip of the plurality of sensor tips to be advanced to the coupler; and
a disposal container configured to capture the sensor tip after use.

20. The sample capture and testing system of claim 18, further comprising:
at least one buffer station;
at least one rinse station; and
a carousel configured to hold the plurality of sensor tips and rotate the plurality of sensor tips from the specimen container holder sequentially to a buffer station of the at least one buffer station to a rinse station of the at least one rinse station and return to the specimen container holder as the bar is applied in subsequent operations.

21. The sample capture and testing system of claim 18, further comprising:
a specimen container;
a removable specimen funnel; and
a specimen cap for coupling to the specimen container and receiving the sensor tip when the specimen container is disposed in the specimen container holder.
